(19) United States
(12) Patent Application Publication
(10) Pub. No.: US 2024/0026314 A1
(43) Pub. Date: Jan. 25, 2024

(54) POLYPEPTIDES FOR USE IN THE SYNTHESIS OF BIOACTIVE PHENOLIC COMPOUNDS

(71) Applicants: Steven ROTHSTEIN, Guelph (CA); Tariq AKHTAR, Guelph (CA); Jose CASARETTO, Guelph (CA); Colby PERRIN, Guelph (CA); Kristen VAN GELDER, Guelph (CA)

(72) Inventors: Steven ROTHSTEIN, Guelph (CA); Tariq AKHTAR, Guelph (CA); Jose CASARETTO, Guelph (CA); Colby PERRIN, Guelph (CA); Kristen VAN GELDER, Guelph (CA)

(21) Appl. No.: 18/254,593

(22) PCT Filed: Nov. 24, 2021
(86) PCT No.: PCT/CA2021/051682

§ 371 Date: May 25, 2023

Related U.S. Application Data
(60) Provisional application No. 63/118,428, filed on Nov. 25, 2020.

(51) Int. Cl. C12N 9/10; C12P 7/22; C12N 15/63; A61K 8/34

(52) U.S. Cl. CPC C12N 9/1085; C12N 9/1011; C12P 7/22; C12N 15/63; A61K 8/347

(57) ABSTRACT

Described herein is a polypeptide encoding a prenyltransferase for prenylating a polyphenol and a polypeptide encoding an O-methyltransferase for methylating a polyphenol. For example, the polypeptide comprises or consists of the sequence of SEQ ID NO: 1, 2, 3, 4, 5, and/or 6, and/or a polypeptide listed in Table 1, and/or SEQ ID NO: 7-30, or a variant thereof having at least 80% sequence identity to SEQ ID NO: 1, 2, 3, 4, 5, and/or 6, and/or the polypeptide listed in Table 1, and/or SEQ ID NO: 7-30, or a fragment of the polypeptide or the variant thereof.

### FIELD

The present invention relates to polypeptides. More specifically, the present invention is, in embodiments, concerned with the use of polypeptides encoding enzymes capable of modifying phenolic molecules and related products, methods, and uses.

### BACKGROUND

Natural product compounds of the polyphenol class often possess anti-oxidant, anti-inflammatory, anti-microbial, anti-viral, and anti-cancer activities (Kikuchi et al. 2019; Kumar and Pandey 2013; Rasouli et al. 2017). Production of these compounds relies on the phenylpropanoid pathway which starts with the synthesis of hydroxycinnamic acids, a C6-C3 carbon backbone synthesized from either phenylalanine or tyrosine. From them, flavonoids (C6-C3-C6) and stilbenes or bibenzyls (C6-C2-C6) can be synthesized. Because of their wide distribution in plants and their health-promoting properties, flavonoids are a well-studied group of compounds. In plants, flavonoids, including chalcones, flavanones, flavones, flavonols and isoflavones, share a basic flavan structure of 15 carbon atoms derived from a C6-C3-C6 skeleton. Modification of primary and secondary metabolites by glycosylation, methylation or prenylation is one of nature's means to modulate their bioactivity and contributes substantially to the large diversity of secondary metabolites present in plants, fungi, and bacteria. Therefore, it is not surprising that flavonoids are usually found naturally as derivatives, e.g., glycosides, methyl ethers and prenylated forms (Koirala et al. 2016; Xiao et al. 2014; Yang et al. 2015).

Over 1000 natural products with one or more prenyl groups have been isolated predominantly from higher plants. Prenylation has been detected on most flavonoids, though prenylated flavonones are the most common subclass and prenylated flavanols are the rarest subclass, with C-prenylation being much more common than 0-prenylation. C-prenylation occurs more frequently on ring A at C-6/C-8 positions and on ring B at C-3′ and C-5′. Prenylation at ring C is rare in natural prenylated flavonoids. In terms of prenyl groups, 3,3-dimethylallyl group (5 C) is the most common example presented in nature, though geranyl (C and farnesyl (C 15) flavonoids are also well known in prenylated flavonoids (Barron & Ibrahim, 1996).

In plants, O-methylated flavonoids (or methoxyflavonoids) are more widely distributed than the C-methylated forms, and the methoxyl group is often present on the C-2′, 3′, 4′, 5′, 3, 5, 6, 7, and 8 positions of flavonoids (Bandyukova and Avanesov, 1971). Because methylated forms of flavonoids present higher metabolic stability and increased membrane transport in the intestine and liver than the hydroxylated counterparts, resulting in greater oral bioavailability, it has been suggested that they also have stronger anticancer potential (Bernini et al. 2011; Walle 2007).

In the case of prenylated plant polyphenols, studies on chemical structures revealed that prenylation enhances their biological activity compared with those non-prenylated forms. For example, the prenylated phenylpropanoids drupanin, artepillin C and baccharin, which are p-coumaric acid derivatives, have been shown to induce apoptotic events in a colon cancer cell line SW480 and human leukemia cell line HL60 (Akao et al 2008); and only oral administration of these prenylated phenylpropanoids causes a significant reduction in tumor growth to mice with sarcoma S-180 (Mishima et al 2005). The mechanism for their enhanced biological activities relies on better membrane permeability due to the lipophilicity of the prenyl moiety, whereby they engage in improved interaction with biological targets such as cell membranes, transporters and other proteins. (Maitrejean et al. 2000; Murakami et al. 2000).

Stilbenoids and dihydrostilbenoids (bibenzyls), which are usually classified as phytoalexins, are antimicrobial compounds used by plants to protect themselves against fungal infection and toxins. Similar to flavonoids, many stilbenoids, such as the recognized resveratrol, pterostilbene, and piceatannol, have important biological effects. Among their biological activities, they have been shown to offer promise in cancer prevention and treatment, cardioprotection, neuroprotection, anti-diabetic properties, and anti-inflammation (Akinwumi et al. 2018; Xiao et al. 2008).

Actinobacteria is a large phylum of terrestrial and aquatic Gram-positive bacteria. Their main representative genera are a source of many antibiotics. Soluble prenyltransferases presenting relaxed substrate specificity and displaying regiospecificity in prenyl group transfer and prenyl chain selectivity have been identified in Actinobacteria, especially among the Streptomyces genus (Bonitz et al. 2011). For example, in Streptomyces sp. CL190, biosynthesis of the anti-oxidant naphterpin includes prenylation of flaviolin with a geranyl group by the prenyltransferase NphB. This enzyme was shown to have broad substrate specificity, and also is able to prenylate several plant polyphenols including 1,6-dihydroxpaphthalene (DHN), naringenin, daidzein, genistein, resveratrol, and olivetol (Kuzuyama et al. 2005). Interestingly, other genes encoding prenyltransferases (namely SCO7190, NovQ, and CloQ) have also been identified from other Streptomyces species (S. coelicolor A3, S. niveus, respectively) and also show loose substrate specificity (Kumano et al. 2008; Ozaki et al. 2009).

U.S. Patent Application Publication No. 2006/0183211 describes a novel aromatic prenyltransferase, Orf2 from Streptomyces sp. strain CL190, involved in naphterpin biosynthesis. This prenyltransferase catalyzes the formation of a C—C bond between a prenyl group and a compound containing an aromatic nucleus and also displays C—O bond formation activity. Numerous crystallographic structures of the prenyltransferase have been solved and refined and provide a mechanistic basis for understanding prenyl chain length determination and aromatic co-substrate recognition in this structurally unique family of aromatic prenyltransferases.

U.S. Patent Application Publication No. 2019/0352679 describes the use of enzyme combinations or recombinant microbes comprising the same to make isoprenoid precursors, isoprenoids and derivatives thereof including prenylated aromatic compounds. Novel metabolic pathways exploiting Claisen, aldol, and acyioin condensations are used instead of the natural mevalonate (MVA) pathway or 1-deoxy-d-xylulose 5-phosphate (DXP) pathways for generating isoprenoid precursors such as isopentenyl pyrophosphate (IPP), dimethylallyl pyrophosphate (DMAPP), and geranyl pyrophosphate (GPP).

U.S. Patent Application Publication No. 2006/0137207 describes novel flavonoid compounds having antioxidant activity. The compounds have been shown to exhibit anti-oxidative properties in biological systems and their utility in a sunscreen or skincare composition or to treat conditions involving oxidative damage, especially curative or prophylactic treatment of Alzheimer's disease or ischaemia-reperfusion injury, is described.

U.S. Patent Application Publication No. 2019/0100549 describes compounds useful in the treatment of many diseases such as a skin disease, an allergy, an autoimmune disease, a cardiovascular disease, a lung disease, asthma, a bacterial, viral or parasitic disease, metabolic syndrome, cancer, Alzheimer's disease or diabetes and are furthermore useful in the preparation of cosmetics and for use in food and animal feed.

U.S. Patent Application Publication No. 2018/0135029 describes a method for producing flavonoids, comprising the steps: (a) providing of a transgenic microorganism, containing (i) a first nucleic acid section (A), comprising or consisting of a gene coding for a CYP450 oxidase, (ii) a second nucleic acid section (B), comprising or consisting of a gene coding for a plant O-methyltransferase, and (b) adding of one or more flavanones to the transgenic microorganism, (c) the conversion of the substrate flavanones by the transgenic microorganism to the corresponding flavonoids, and optionally (d) isolating and purifying of the final products.

U.S. Patent Application Publication No. 2007/0150984 describes a genetic sequence encoding a polypeptide having methyltransferase activity and the use of the genetic sequence and/or the polypeptide to modify one or more phenotypic characteristics of a plant. More particularly, the methyltransferase of the present invention acts on flavonoids, preferably wherein the flavonoid is an anthocyanin. Described is a polypeptide having S-adenosyl-L-methionine:anthocyanin 3′-O-methyl-transferase or S-adenosyl-L-methionine:anthocyanin 3′,5′-O-methyltransferase activity. Further described is a genetic sequence encoding a polypeptide having methyltransferase activity derived from Petunia, Torenia Fuchsia or Plumbago or botanically related plants.

U.S. Patent Application Publication No. 2016/0273006 describes a biosynthetic method of making pterostilbene including expressing a 4-coumaratexoenzyme A ligase (4CL) in a cellular system, expressing a stilbene synthase (STS) in the cellular system, expressing a resveratrol O-methyltransferase (ROMT) in the cellular system, feeding p-coumaric acid to the cellular system, growing the cellular system in a medium, and producing pterostilbene.

U.S. Pat. No. 7,732,666 relates to an O-methyltransferase gene cloned from sorghum, the sorghum O-methyltransferase-3 gene, SbOMT3. Quantitative real-time RT-PCR and recombinant enzyme studies with putative O-methyltransferase sequences obtained from an EST data set from sorghum have led to the identification of the novel root hair-specific O-methyltransferase designated SbOMT3. Transgenic plants which express SbOMT3 can convert resveratrol into pterostilbene in planta. SbOMT3 is also involved in the biosynthesis of sorgoleone.

There is a need to provide a useful alternative to overcome at least some of the deficiencies of the prior art.

### DESCRIPTION OF THE DRAWINGS

The present invention will be further understood from the following description with reference to the Figures:

FIG. 1. Evidence for cannflavin A synthesis by NphB. (A) Representative HPLC chromatogram for an authentic cannflavin A standard. (B) Representative chromatogram of the product from a cell-free enzyme assay with recombinant NphB plus chrysoeriol and GPP. Note that the assay produces two major products; the first major peak elutes at the same time as the cannflavin A standard while the second peak elutes approximately 1 min later. The first major peak (“1”) which corresponds to cannflavin A elution time was collected and processed for mass spectrometry analysis.

FIG. 2. Mass spectrometry evidence for the synthesis of cannflavin A from chrysoeriol by NphB. (A) Q-TOF mass spectra of a cannflavin A standard (upper panel). The lower panel shows the collision-induced dissociation (CID)-Q-TOF mass spectral fragmentation pattern of such standard. (B) The first peak, which corresponds to an enzymatic product with the same retention time as cannflavin A on the HPLC (FIG. 1B), was collected offline. The upper panel shows that the mass spectrum of such a product (6-geranyl chrysoeriol) is consistent with the pattern of a cannflavin A standard ([M+H]+ 437). The CID-Q-TOF mass spectral fragmentation pattern of the enzymatic product from this assay (bottom panel) also resembles that of the cannflavin A standard in (A, bottom panel), indicating that prenylation of chrysoeriol with GPP by NphB produces cannflavin A.

FIG. 3. Selective methylation of flavonoids by O-methyltransferases form Cannabis. (A) Three flavones (apigenin, luteolin and chrysoeriol) and two flavonols (quercetin and kaempferol) that typically accumulate in C. sativa were tested as potential substrates for CsOMT6 and CsOMT21. Relative enzymatic activity and substrate preference of (B) CsOMT6 and (C) CsOMT21. The selected flavones and flavonols (as numbered in A) were provided to recombinant CsOMT6 or CsOMT21 that were purified by Ni2+-affinity chromatography in enzyme assays along with [¹⁴C]-SAM as a methyl donor. Data are means±SD from three independent experiments and are presented as relative activity compared to that observed with the preferred substrate for each enzyme.

FIG. 4. Methylation of bibenzyls by O-methyltransferases from Cannabis. (A) Four bibenzyl compounds (dihydroresveratrol, tristin, gigantol and batatasin III) were tested as potential substrates for CsOMT1, CsOMT3, CsOMT5, and CsOMT13. (B) Relative enzymatic activity of the four OMTs. The four selected bibenzyl compounds were provided to purified recombinant CsOMT1, CsOMT3, CsOMT5 or CsOMT13 in enzyme assays along with [¹⁴C]-SAM as a methyl donor. Data are means from three independent experiments and are shown as relative activity compared to that of CsOMT1 with dihydroresveratrol.

FIG. 5. Selective methylation of a bibenzyl by CsOMT1. (A) Phenolic compounds representing a bibenzyl (dihydroresveratrol), stilbenoids (resveratrol and pinosylvin), and a hydroxycinamic acid (caffeic acid) and in its reduced form (dihydrocaffeic acid) were tested as substrates for CsOMT1. (B) Relative enzymatic activity and substrate preference of CsOMT1. The five compounds were provided to purified recombinant CsOMT1 in enzyme assays along with [¹⁴C]-SAM as a methyl donor. Data are means from three independent experiments and are shown as relative activity compared to that of CsOMT1 with dihydroresveratrol.

FIG. 6. Enzymatic methylation activity of CsOMT1. (A) Reaction catalyzed by CsOMT1. (B) Representative chromatogram showing the reaction products resolved by HPLC that illustrates the separation of the methylated dihydroresveratrol from its corresponding substrate (dihydroresveratrol, DHR). The identity of 3-O-methyl-dihydroresveratrol (pinobistilbene) was structurally determined by NMR. (C) Kinetic analysis of CsOMT1. Recombinant CsOMT1 was assayed under standard assay conditions at the indicated concentrations of dihydroresveratrol. Kinetic parameters were determined by non-linear regression analysis using the Michaelis-Menten kinetics model of the SigmaPlot 12.3 software.

### SUMMARY

In accordance with an aspect, there is provided a polypeptide encoding a prenyltransferase for prenylating a polyphenol.

In an aspect, the prenyltransferase is a microbial prenyltransferase.

In an aspect, the polypeptide is comprising or consisting of a polypeptide having at least 80%, 85%, 90%, 91%, 92%, 93%, 94%, 95%, 96%, 97%, 98%, 99%, or 100% sequence identity to the sequence of any one or more of SEQ ID NO: 1-6 and/or a polypeptide listed in Table 1, or a fragment of any thereof.

In an aspect, the polypeptide is comprising or consisting of the sequence of any one or more of SEQ ID NO: 1-6 and/or a polypeptide listed in Table 1.

In an aspect, the polypeptide is comprising or consisting of the sequence of any one or more of SEQ ID NO: 1-6.

In an aspect, the polypeptide prenylates the polyphenol using a prenyl donor.

In an aspect, the prenyl donor is IPP, FPP, GPP, and/or DMAPP, or a variant or derivative thereof.

In an aspect, the polyphenol is a flavonoid, stilbenoid, and/or bibenzyl, or a derivative thereof.

In an aspect, the flavonoid is a flavone, such as apigenin, luteolin, chrysoeriol, chrysin, acacetin, baicalein, baicalin, vitexin, wogonin, orientin, oroxylin A. rutin, or tangeritin; a flavonol such as quercetin, kaempferol, galangin, myricetin, tamarixetin, fisetin, or casticin; a flavanone such as naringenin, hesperetin, pinocembrin, hesperidin, or eriodictyol; a flavanonol such as taxifolin; a flavanol such as catechin, or epicatechin; an isoflavone such as genistein, or daidzein; an anthocyanin such as cyanidin, chrysanthemin, pelargonidin, delphinidin, or malvidin; or any combination thereof.

In an aspect, the stilbenoid is resveratrol, piceatannol, pterostilbene, pinosylvin, gnetol, oxyresveratrol, pinostilbene, or any combination thereof.

In an aspect, the bibenzyl is a dihydrostilbenoid such as dihydroresveratrol, combretastatin, dihydropiceatannol, dihydrognetol, dihydropinosylvin, gigantol, pinobistilbene, batatasin III, crepidatin, moscatilin, crysotoxine, chrysotobibenzyl, amoenylin, tristin, cumulating, or any combination thereof.

In an aspect, the prenyltransferase prenylates the flavonoid to produce 8-prenyl kaempferol, isocannflavin B, cannflavin C, 6-prenylnaringenin, 6-prenylapigenin, neougonin A, neougonin B, and/or kuraridin.

In an aspect, the prenyltransferase prenylates the stilbenoid to produce arachidins, isorhapontigenin, rhapontigenin, pawhuskin A, aglaiabbrevin E, amorphastilbol, or longistylins.

In an aspect, the prenyltransferase prenylates the bibenzyl to produce canniprene, cannabistilbene, dihydrolongistylins, amorfrutin 1/A, or amorfrutin B.

In an aspect, the prenyltransferase prenylates chrysoeriol using GPP to produce cannflavin A.

In an aspect, the prenyltransferase prenylates chrysoeriol using DMAPP to produce cannflavin B.

In accordance with an aspect, there is provided a polypeptide encoding an O-methyltransferase for methylating a polyphenol.

In an aspect, the O-methyltransferase is a plant O-methyltransferase.

In an aspect, the O-methyltransferase is a Cannabis sativa O-methyltransferase.

In an aspect, the polypeptide is comprising or consisting of a polypeptide having at least 80%, 85%, 90%, 91%, 92%, 93%, 94%, 95%, 96%, 97%, 98%, 99%, or 100% sequence identity to the sequence of any one or more of SEQ ID NO: 7-30, or a fragment of any thereof.

In an aspect, the polypeptide is comprising or consisting of the sequence of any one or more of SEQ ID NO: 7-30.

In an aspect, the polypeptide methylates the polyphenol using a methyl donor.

In an aspect, the methyl donor is S-adenosyl methionin, or a variant or derivative thereof.

In an aspect, the polyphenol is a flavonoid, stilbenoid, and/or bibenzyl, or a derivative thereof.

In an aspect, the flavonoid is a flavone, such as apigenin, luteolin, chrysoeriol, chrysin, acacetin, baicalein, baicalin, vitexin, wogonin, orientin, oroxylin A. rutin, or tangeritin; a flavonol such as quercetin, kaempferol, galangin, myricetin, tamarixetin, fisetin, or casticin; a flavanone such as naringenin, hesperetin, pinocembrin, hesperidin, or eriodictyol; a flavanonol such as taxifolin; a flavanol such as catechin, or epicatechin; an isoflavone such as genistein, or daidzein; an anthocyanin such as cyanidin, chrysanthemin, pelargonidin, delphinidin, or malvidin; or any combination thereof.

In an aspect, the stilbenoid is resveratrol, piceatannol, pterostilbene, pinosylvin, gnetol, oxyresveratrol, or any combination thereof.

In an aspect, the bibenzyl is a dihydrostilbenoid such as dihydroresveratrol, combretastatin, dihydropiceatannol, dihydrognetol, dihydropinosylvin, gigantol, batatasin III, crepidatin, moscatilin, crysotoxine, chrysotobibenzyl, amoenylin, tristin, cumulating, or any combination thereof.

In an aspect, the O-methyltransferase methylates a flavonoid to produce chrysoeriol, acacetin, tamarixetin, or methylquercetin.

In an aspect, the O-methyltransferase methylates a stilbenoid to produce pinostilbene, isorhapontigenin, rhapontigenin, or any combination thereof.

In an aspect, the O-methyltransferase methylates a bibenzyl to produce gigantol, tristin, orpinobistilbene.

In an aspect, the polypeptide, variant, or fragment comprises up to about 100, about 150, about 200, about 250, about 300, about 350, about 360, about 370, about 380, about 390, about 400, about 410, about 420, about 430, about 440, about 450, or about 500 amino acids

In an aspect, the polypeptide is synthetic.

In an aspect, the polypeptide is recombinant.

In accordance with an aspect, there is provided a nucleic acid encoding the polypeptide described herein.

In an aspect, the nucleic acid is cDNA.

In accordance with an aspect, there is provided a vector comprising the nucleic acid described herein.

In accordance with an aspect, there is provided a host cell comprising the vector described herein.

In accordance with an aspect, there is provided a host cell expressing the polypeptide described herein.

In an aspect, the host cell is a bacterial cell (e.g., E. coli or Agrobacterium tumefaciens), a yeast cell (e.g., S. cerevisiae), an algal cell, or a plant cell (e.g., Nicotiana spp.).

In an aspect, the host cell is provided in combination with the polyphenol.

In an aspect, the polyphenol is provided in the host cell culture medium.

In an aspect, the polyphenol is expressed by the host cell.

In an aspect, the host cell is provided in combination with a prenyl donor and/or a methyl donor.

In an aspect, the prenyl donor and/or methyl donor is provided in the host cell culture medium.

In an aspect, the prenyl donor and/or methyl donor is expressed by the host cell.

In accordance with an aspect, there is provided an expression system comprising the polypeptide; the nucleic acid, the vector; or the host cell described herein.

In an aspect, the expression system further comprises the polyphenol and a prenyl donor and/or methyl donor.

In accordance with an aspect, there is provided a system for prenylating and/or methylating a polyphenol the system comprising the polypeptide described herein.

In an aspect, the polypeptide is in a batch solution.

In an aspect, the polypeptide is immobilized in a support matrix.

In an aspect, the polypeptide is in a cell.

In an aspect, the system is cell-free.

In accordance with an aspect, there is provided a method for prenylating and/or methylating a polyphenol, wherein the method comprises contacting the polyphenol with the polypeptide described herein.

In an aspect, the method is carried out in the system described herein.

In an aspect, the method is a recombinant method comprising expressing the polypeptide described herein in a cell in the presence of the polyphenol and a prenyl donor and/or methyl donor.

In an aspect, the method is in combination with a synthetic chemical catalysis method.

In an aspect, the method comprises a single synthesis step.

In an aspect, the method is carried out in combination with an enzymatic reaction.

In an aspect, the method comprises a combined enzymatic O-methylation and prenylation step.

In accordance with an aspect, there is provided a method of producing cannflavin A, cannflavin B, isocannflavin B, the method comprising carrying out a combined enzymatic O-methylation and prenylation of a flavonoid.

In accordance with an aspect, there is provided a method of producing a longistylin, the method comprising carrying out a combined enzymatic O-methylation and prenylation of a stilbenoid.

In accordance with an aspect, there is provided a method of producing canniprene, cannabistilbene, dihydrolongistylin, amorfrutin 1/A, or amorfrutin B, the method comprising carrying out a combined enzymatic O-methylation and prenylation of a bibenzyl.

In accordance with an aspect, there is provided a synthetic chemical catalysis method of producing cannflavin A and/or cannflavin B, the method comprising using GPP and DMAPP in a single synthesis step from chrysoeriol or in combination with an enzymatic reaction such as the O-methylation of luteolin.

In accordance with an aspect, there is provided a prenylated and/or methylated polyphenol produced by the method described herein.

In an aspect, the polyphenol is substantially pure, for example, at least about 95%, about 96%, about 97%, about 98%, about 99%, about 99.5%, or about 99.9% pure.

In an aspect, the polyphenol is cannflavin A and/or cannflavin B.

In accordance with an aspect, there is provided a cosmetic composition comprising the polyphenol described herein and at least one cosmetically acceptable carrier.

In accordance with an aspect, there is provided a pharmaceutical composition comprising the polyphenol described herein and at least one pharmaceutically acceptable carrier.

In accordance with an aspect, there is provided a natural health product comprising the polyphenol described herein, such as a supplement, beverage, or food.

In accordance with an aspect, there is provided a use of the polyphenol described herein in a cosmetic, pharmaceutical, or natural health product.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### DETAILED DESCRIPTION

The enhanced and distinct biological activities of modified polyphenols have prompted the utilization of plants containing these as medicinal plants and also as ingredients in products for food industries, breweries, and cosmetic companies. Some show promise as important compounds for the development of nutraceuticals and new pharmacological agents for the treatment of different medical conditions. However, there are several obstacles that have limited the potential applications of these compounds:
In many cases, prenylated and/or methylated flavonoids often exist at trace levels in natural sources or are present in a limited number of plant species.
Plant extraction of polyphenols often poses a significant challenge for cost-effective production due to the high cost associated with downstream processing and purification from complicated mixtures. Isolation from native plant sources also depends on external factors (e.g. agricultural, climate and geographic variations), which results in inconsistent quality needed for drug ingredients.
In vivo synthesis in heterologous plant or microbial systems is usually regarded as not sustainable because of low production rates.
Genetic reconstruction of biosynthetic routes raises several challenges in terms of the assembly of a genetic pathway. Although yeast has proven to be an amenable organism for replicating biosynthesis of various plant pathways, only a few have been reconstructed successfully (Paddon and Keasling 2014; Thodey et al. 2014). For example, compartmentalization, expression of integral membrane-bound plant enzymes, branched pathways and specific regulatory mechanisms affecting synthesis are among the major challenges to microorganisms for producing secondary natural products.
In many cases, organic chemical synthesis is not an amenable cost-effective approach.

Described herein are prenyltransferases and methyltransferases that can serve as alternate production catalysts for the in vitro and in vivo prenylation and/or methylation of natural products of plant origin, such as polyphenols, such as flavonoids, stilbenoids and bibenzyls. In addition, the approaches described herein provide the basis for exploring novel prenylation and methylation chemistry and bioactivity of natural and novel synthetic prenylated or methylated aromatic compounds by means of structure-based enzyme engineering.

Thus, described herein are polypeptides and related methods for the synthesis of polyphenols using prenyltransferases and methyltransferases or variants of such enzymes. These are typically derived from microbial, such as bacterial or fungal, or plant sources, such as Cannabis sativa. The use of enzymes with potential prenyltranferase activity is described herein, for example, NphB, HypSc, CloQ, NovQ, Fur7, and PpzP and related sequences. In vitro catalysis systems, designed to utilize these enzymes and thereby produce substantially pure prenylated polyphenols, such as flavonoids, stilbenoids or bibenzyls, are described. For example, NphB or an enzyme with a similar amino acid sequence which prenylates chrysoeriol using GPP or DMAPP to produce cannflavin A or cannflavin B, respectively.

Like cannflavin A and cannflavin B, these and other prenylated flavonoids (for example, 8-prenyl kaempferol, isocannflavin B, cannflavin C, 6-prenylnaringenin, 6-prenylapigenin, neougonin A and B), stilbenoids (for example, pawhuskin A aglaiabbrevin E, amorphastilbol, longistylins) or bibenzyls (for example, canniprene, cannabistilbene, dihydrolongistylins, amorfrutin 1/A, amorfrutin B) may find use in anti-inflammatory compositions and methods.

In addition, the use of plant enzymes with O-methyltransferase activity is described, for example CsOMT1-24. Methylation by these methyltransferases can produce pure polyphenols such as flavonoids (for example, chrysoeriol, acacetin, tamarixetin, methylquercetin), stilbenoids, or bibenzyls (for example gigantol and pinobistilbene), bioactive compounds that can find direct use or can be employed as substrates for further prenylation through an in vitro method or by chemical synthesis.

### Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning. A Laboratory Manual, Cold Springs Harbor Press (Cold Springs Harbor, N Y 1989), each of which is incorporated herein by reference. For the purposes of the present invention, the following terms are defined below.

As used herein, the term “flavonoid” includes, for example, flavanone, flavone, flavonol, flavanonol, isoflavone, anthocyan, and chalcone, as well as derivatives thereof, such as prenylated or methylated derivatives thereof, unless otherwise specified. Examples of flavonoids include naringenin, apigenin, luteolin, myricetin, quercetin, catechin, daidzein, genistein, kaempferol, pelargonidin, delphinidin, and cyanidin. Flavonoids may be of natural or synthetic origin and have the following general structure. Numbers in the ring structures indicate the positions of hydroxyl groups and where modifications such as prenylation, methylation or glycosylation can occur. Usually, the position of such modifications and hydroxyl groups determine than bioactivity of the molecule.

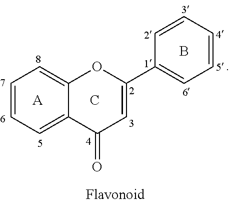

The term “stilbenoid” includes any hydroxylated derivatives of stilbene, as well as derivatives thereof, such as methylated or prenylated derivatives thereof unless otherwise specified. Examples of stilbenoids include resveratrol, piceatannol, pterostilbene, pinosylvin and gnetol. Stilbenoids may be of natural or synthetic origin and have the following general structure. Numbers in the ring structures indicate the positions of hydroxyl groups and where modifications such as prenylation, methylation or glycosylation can occur. Usually, the position of such modifications and hydroxyl groups determines than bioactivity of the molecule.

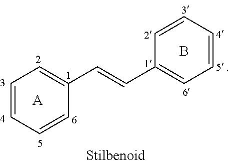

The term “bibenzyl” includes any dihydrostilbene derivatives, as well as derivatives thereof, such as methylated or prenylated derivatives thereof unless otherwise specified. Examples of bibenzyls include dihydroresveratrol, combretastatin, dihydropiceatannol, gigantol, tristin, batatasin Ill, crepidatin, and amoenylin. Bibenzyls may be of natural or synthetic origin and have the following general structure. Numbers in the ring structures indicate the positions of hydroxyl groups and where modifications such as prenylation, methylation or glycosylation can occur. Usually, the position of such modifications and hydroxyl groups determines than bioactivity of the molecule.

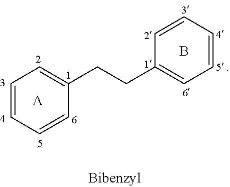

“Variants” of the sequences described herein are biologically active sequences that have a peptide sequence that differs from the sequence of a native or wild-type sequence, by virtue of an insertion, deletion, modification and/or substitution of one or more amino acids within the native sequence. Such variants generally have less than 100% sequence identity with a native sequence. Ordinarily, however, a biologically active variant will have an amino acid sequence with at least about 70% sequence identity with the sequence of a corresponding naturally occurring sequence, typically at least about 75%, more typically at least about 80%, even more typically at least about 85%, even more typically at least about 90%, and even more typically of at least about 95%, 96%, 97%, 98%, or 99% sequence identity. The variants nucleotide fragments of any length that retain a biological activity of the corresponding native sequence. Variants also include sequences wherein one or more amino acids are added at either end of, or within, a native sequence. Variants also include sequences where a number of amino acids are deleted and optionally substituted by one or more different amino acids.

As used herein, “treatment” or “therapy” is an approach for obtaining beneficial or desired clinical results. For the purposes described herein, beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. “Treatment” and “therapy” can also mean prolonging survival as compared to expected survival if not receiving treatment or therapy. Thus, “treatment” or “therapy” is an intervention performed with the intention of altering the pathology of a disorder. Specifically, the treatment or therapy may directly prevent, slow down or otherwise decrease the pathology of a disease or disorder such as inflammation, or may render the inflammation more susceptible to treatment or therapy by other therapeutic agents.

The terms “therapeutically effective amount”, “effective amount” or “sufficient amount” mean a quantity sufficient, when administered to a subject, including a mammal, for example, a human, to achieve a desired result, for example, an amount effective to treat inflammation. Effective amounts of the polyphenolic compounds described herein may vary according to factors such as the disease state, age, sex, and weight of the subject. Dosage or treatment regimens may be adjusted to provide the optimum therapeutic response, as is understood by a skilled person.

Likewise, an “effective amount” of the polyphenolic compounds described herein refers to an amount sufficient to function as desired, such as to treat inflammation.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

“Carriers” as used herein include cosmetically or pharmaceutically acceptable carriers, excipients, or stabilizers that are non-toxic to the cell or subject being exposed thereto at the dosages and concentrations employed. Often the pharmaceutically acceptable carrier is an aqueous pH buffered solution. Examples of pharmacologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, and dextrins; chelating agents such as EDTA; sugar alcohols such as mannitol and sorbitol; salt-forming counterions such as sodium; and/or non-ionic surfactants such as TWEEN™, polyethylene glycol (PEG), and PLURONICS™.

In understanding the scope of the present application, the articles “a”, “an”, “the”, and “said” are intended to mean that there are one or more of the elements. Additionally, the term “comprising” and its derivatives, as used herein, is intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any embodiments described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation. For example, in embodiments, THC, cannabinoids, and/or terpenes are explicitly excluded from the compositions and methods described herein.

In addition, all ranges given herein include the end of the ranges and also any intermediate-range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Polypeptides

Described herein, in aspects, are polypeptides encoding enzymes that are typically prenyltransferases. These polypeptides are typically derived from microbes such as bacteria or fungus and find use in converting polyphenols, such as flavonoid compounds, into compounds with medicinal activity and/or precursors to such compounds. For example, described herein are polypeptides comprising one or more of the following sequences, variants thereof, or fragments of the polypeptides or variants. Variants can be of natural origin such as orthologous sequences from related bacterial or fungal species, or of non-natural versions obtained synthetically or by mutations. In the following sequences, amino acid sequences and GenBank accession numbers of these enzymes and their relatives are shown. Also described herein are orthologous sequences similar to SEQS NO:1-6 found in nature with their respective GenBank accession numbers (see Table 1).

>NphB/Orf2/BAE00106 [Streptomyces sp. CL190]
SEQ ID NO: 1
MSEAADVERVYAAMEEAAGLLGVACARDKIYPLLSTFQDTLVEGGSVVV
ESMASGRHSTELDESISVPTSHGDPYATVVEKGLFPATGHPVDDLLADT
QKHLPVSMFAIDGEVTGGFKKTYAFFPTDNMPGVAELSAIPSMPPAVAE
NAELFARYGLDKVQMTSMDYKKRQVNLYESELSAQTLEAESVLALVREL
GLHVPNELGLKFCKRSFSVYPTLNWETGKIDRLCFAVISNDPTLVPSSD
EGDIEKFHNYATKAPYAYVGEKRTLVYGLTLSPKEEYYKLGAYYHITDV
QRGLLKAFDSLED
>HypSc/SCO7190/NP631248
[Streptomyces coelicolor A3 (2)]
SEQ ID NO: 2
MPTGRTTDLALFLSDLEAYAKLAEVTEDKRAVEQVVDVEAEQFATGTIT
VRTTTHEAANRSVNFRYMYPDSPHDPVEIARAHGLLPDADPAVMSLLAE
VTEKIPLWWGLDASVGHGVQKVWAFFEQPLEFGEIASLENTPHSLRDHR
ERFGEARIDRFAIMGFDERDNTTNLYSEMVSPGYFEQEEVARMIRDVGS
LPPDNEEIERCRGAINVYYTEDWNSPQARRLCFAVPSRDGEFPSHLHPL
AARFAAEAPVQAERRELIENPTFGARGSYLKMEADYTGDAASRVEGYWN
R
>CloQ/AAN65239 [Streptomyces roseochromogenus
subsp. oscitans]
SEQ ID NO: 3
MPALPIDQEFDCERFRADIRATAAAIGAPIAHRLTDTVLEAFRDNFAQG
ATLWKTTSQPGDQLSYRFFSRLKMDTVSRAIDAGLLDAAHPTLAVVDAW
SSLYGGAPVQSGDFDAGRGMAKTWLYFGGLRPAEDILTVPALPASVQAR
LKDELALGLAHVRFAAVDWRHHSANVYFRGKGPLDTVQFARIHALSGST
PPAAHVVEEVLAYMPEDYCVAITLDLHSGDIERVCFYALKVPKNALPRI
PTRIARFLEVAPSHDVEECNVIGWSFGRSGDYVKAERSYTGNMAEILAG
WNCFFHGEEGRDHDLRALHQHTESTMGGAR
>NovQ/AAF67510 [Streptomyces niveus]
SEQ ID NO: 4
MPALPMNQEFDRERFRVDLRATAAAIGAPVTPRVTDTVLETFRDNFAQG
ATLWKTTSQPGDQLSYRFFSRLKMDTVGRAVDAGLLDGTHPTVPIVEDW
SDLYGGTPVQSADFDAGRGMAKTWLYFGGLRPAEDILSVPALPAPVQAR
LKDELGLGLAHVRFAAVDWRHRSANVYFRGQGPLDTAQFARVHALSGGT
PPAADVVAEVLAYVPEDYCVAITLDLHTGAIDRVCFYALKVPKDARPRV
PARIATFLEVAPSHDPEECNVIGWSFGRSGDYVKAERSYTGNMTEILSG
WNCFFHGEEGRDHDLRALQDTGSITGGAR
>Fur7/Q2L6E3.1 [Streptomyces sp. KO-3988]
SEQ ID NO: 5
MPGTDDVAVDVASVYSAIEKSAGLLDVTAAREVVWPVLTAFEDVLEQAV
IAFRVATNARHEGDEDVRFTVPEEVDPYAVALSRSLIAKTDHPVGSLLS
DIQQLCSVDTYGVDLGVKSGFKKVWVYFPAGEHETLARLTGLTSMPGSL
AGNVDFFTRYGLADKVDVIGIDYRSRTMNVYFAAPSECFERETVLAMHR
DIGLPSPSEQMFKFCENSFGLYTTLNWDTMEIERISYGVKTENPMTFFA
RLGTKVEHFVKNVPYGVDTQKMVYAAVTSSGEEYYKLQSYYRWRSVSRL
NAAYIAARDKEST
>PpzP/C4PWA1. 1 [Streptomyces anulatus]
SEQ ID NO: 6
MSESAELTELYSAIEETTRVVGAPCRRDTVRPILTAYEDVIAQSVISFR
VQTGTSDAGDLDCRFTLLPKDMDPYATALSNGLTAKTDHPVGSLLEEVH
RQFPVDCYGIDFGAVGGFKKAWSFFRPDSLQSASDLAALPSMPSGVSEN
LGLEDRYGMTDTVSVVGEDYAKRSVNLYFTGASPESFEPRGIQAILREC
GLPEPSDELLRFGEEAFAIYVTLSWDSQKIERVTYSVNTPDPMALPVRI
DTRIEQLVKDAPLGSAGHRYVYGVTATPKGEYHKIQKYFQWQSRVEKML
TADAG

TABLE 1
Accessions with significant similarity amino acid sequences to SEQS NO: 1-6

| No | Accession | Description | % Ident. |
|---|---|---|---|
| 1 | WP_057602682.1 | hypothetical protein [Streptomyces sp. Root1310] NphB | 100.00% |
| 2 | 1ZB6_A | Chain A, Aromatic Prenyltransferase [Streptomyces sp.] HypSc | 100.00% |
| 3 | WP_023545098.1 | 4-hydroxyphenylpyruvate 3-DMAPP transferase [Streptomyces roseochromogenus], CloQ | 100.00% |
| 4 | WP_079127921.1 | hypothetical protein [Streptomyces] NovQ | 100.00% |
| 5 | Q2L6E3.1 | Furaquinocin biosynthesis prenyltransferase [Streptomyces sp. KO-3988] Fur7 | 100.00% |
| 6 | C4PWA1.1 | Dihydro-PCA dimethylallyltransferase; [Streptomyces anulatus] PpzP | 100.00% |
| 7 | 2XLQ_A | Chain A, CLOQ [Streptomyces roseochromogenus subsp. oscitans] | 100.00% |
| 8 | 2XM7_A | Chain A, CLOQ [Streptomyces roseochromogenus subsp. oscitans] | 99.69% |
| 9 | WP_069622146.1 | hypothetical protein [Streptomyces niveus] | 98.76% |
| 10 | EST19414.1 | hypothetical protein M877_36355 [Streptomyces niveus NCIMB 11891] | 98.74% |
| 11 | AFS18550.1 | ABBA prenyltransferase Ptf_St [Streptomyces tendae] | 97.38% |
| 12 | WP_078566427.1 | prenyltransferase [Streptomyces sp. CNS654] | 90.82% |
| 13 | WP_109499705.1 | hypothetical protein [Streptomyces sp. Act143] | 89.84% |
| 14 | WP_017621683.1 | hypothetical protein [Nocardiopsis gilva] | 89.81% |
| 15 | KUN17719.1 | hypothetical protein AQJ23_40425 [Streptomyces antibioticus] | 88.93% |
| 16 | KPI30857.1 | Aromatic prenyltransferase, Orf2 [Actinobacteria bacterium OV320] | 83.39% |
| 17 | WP_037872080.1 | aromatic prenyltransferase [Streptomyces sp. NRRL S-37] | 81.64% |
| 18 | WP_114664513.1 | prenyltransferase [Streptomyces sp. GSSD-12] | 81.31% |
| 19 | WP_067123092.1 | prenyltransferase [Streptomyces yokosukanensis] | 80.27% |
| 20 | WP_114021785.1 | prenyltransferase [Candidatus Streptomyces philanthi] | 80.07% |
| 21 | WP_015662653.1 | hypothetical protein [Streptomyces davaonensis] | 78.43% |
| 22 | WP_114016600.1 | prenyltransferase [Streptomyces sp. LHW50302] | 78.43% |
| 23 | GCB50864.1 | hypothetical protein SNL152K_8211 [Streptomyces sp. NL15-2K] | 76.08% |
| 24 | WP_124443591.1 | prenyltransferase [Streptomyces sp. NL15-2K] | 74.58% |
| 25 | WP_078582630.1 | hypothetical protein [Streptomyces sp. URHA0041] | 73.11% |
| 26 | WP_073501789.1 | hypothetical protein [Streptomyces paucisporeus] | 73.11% |
| 27 | WP_078869665.1 | hypothetical protein [Streptomyces sp. NRRL B-1347] | 71.95% |
| 28 | WP_063792633.1 | prenyltransferase [Streptomyces atriruber] | 67.58% |
| 29 | WP_150163952.1 | prenyltransferase [Streptomyces venezuelae] | 66.56% |
| 30 | A2AXG5.1 | Flaviolin linalyltransferase [Streptomyces cinnamonensis], Fnq26 | 66.21% |
| 31 | WP_150181843.1 | prenyltransferase [Streptomyces venezuelae] | 65.89% |
| 32 | WP_101421563.1 | prenyltransferase [Streptomyces sp. CMB-StM0423] | 65.80% |
| 33 | WP_047018069.1 | hypothetical protein [unclassified Streptomyces] | 65.57% |
| 34 | WP_052770383.1 | hypothetical protein [unclassified Streptomyces] | 64.47% |
| 35 | WP_125936269.1 | prenyltransferase [Streptomyces sp. WAC 06738] | 64.14% |
| 36 | WP_101422827.1 | hypothetical protein [Streptomyces sp. CMB-StM0423] | 63.82% |
| 37 | AKH86873.1 | hypothetical protein AA958_18620 [Streptomyces sp. CNQ-509] | 63.57% |
| 38 | WP_063790017.1 | prenyltransferase [Streptomyces sp. MMG1121] | 60.88% |
| 39 | WP_078627616.1 | hypothetical protein [Streptomyces sp. CNH099] | 60.53% |
| 40 | AQU65790.1 | hypothetical protein BBN63_05560 [Streptomyces niveus] | 60.33% |
| 41 | WP_147874937.1 | hypothetical protein [Streptomyces sp. IB2014 016-6] | 60.33% |
| 42 | WP_142270720.1 | prenyltransferase [Streptomyces sp. SLBN-115] | 59.86% |
| 43 | WP_030308521.1 | hypothetical protein [Streptomyces sp. NRRL F-6131] | 59.08% |
| 44 | WP_053649621.1 | hypothetical protein [Streptomyces sp. XY431] | 58.94% |
| 45 | WP_097275187.1 | prenyltransferase [Streptomyces sp. TLI_55] | 58.89% |
| 46 | WP_132804080.1 | prenyltransferase [Streptomyces sp. BK042] | 58.74% |
| 47 | WP_045940576.1 | hypothetical protein [Streptomyces sp. NRRL S-495] | 58.61% |
| 48 | WP_103552886.1 | prenyltransferase [Streptomyces populi] | 58.16% |
| 49 | WP_030397806.1 | hypothetical protein [Kitasatospora] | 58.09% |
| 50 | WP_067237424.1 | prenyltransferase [Streptomyces longwoodensis] | 58.01% |
| 51 | WP_137304658.1 | prenyltransferase [Streptomyces lasaliensis] | 57.95% |
| 52 | WP_093861400.1 | hypothetical protein [Streptomyces sp. TLI_053] | 57.76% |
| 53 | WP_026220570.1 | aromatic prenyltransferase [Streptomyces vitaminophilus] | 57.45% |
| 54 | 4EE8_A | Chain A, Prenyltransferase [Streptomyces cinnamonensis] | 57.10% |
| 55 | ADQ43372.1 | prenyltransferase [Streptomyces cinnamonensis] | 57.10% |
| 56 | WP_130469611.1 | prenyltransferase [Actinopolyspora sp. DSM 45956] | 56.79% |
| 57 | WP_150173938.1 | prenyltransferase [Streptomyces venezuelae] | 56.77% |
| 58 | WP_063784116.1 | prenyltransferase [Streptomyces sp. SBT349] | 56.66% |
| 59 | WP_146478482.1 | prenyltransferase [Streptomyces sp. SSL-25] | 56.00% |
| 60 | RZU69833.1 | aromatic prenyltransferase Orf2 [Actinopolyspora sp. DSM 45956] | 55.99% |
| 61 | 4EE6_A | Chain A, Prenyltransferase [Streptomyces cinnamonensis] | 55.78% |
| 62 | WP_092625972.1 | prenyltransferase [Actinopolyspora mzabensis] | 55.76% |
| 63 | 4EE6_B | Chain B, Prenyltransferase [Streptomyces cinnamonensis] | 55.45% |
| 64 | WP_137303681.1 | prenyltransferase [Streptomyces galbus] | 55.05% |
| 65 | AEW22941.1 | WT5.12c [Streptomyces sp. WT5] | 54.97% |
| 66 | WP_139642050.1 | prenyltransferase [Streptomyces sedi] | 54.85% |
| 67 | WP_027750390.1 | aromatic prenyltransferase [Streptomyces sp. CNH287] | 54.85% |
| 68 | WP_055569452.1 | hypothetical protein [Streptomyces atriruber] | 54.79% |
| 69 | WP_079307203.1 | prenyltransferase [Streptomyces sp. GKU 895] | 54.73% |
| 70 | WP_139674960.1 | prenyltransferase [unclassified Streptomyces] | 54.70% |
| 71 | WP_037700793.1 | aromatic prenyltransferase [Streptomyces atratus] | 54.33% |
| 72 | WP_091645455.1 | prenyltransferase [Micromonospora pallida] | 54.05% |
| 73 | WP_111241504.1 | prenyltransferase [Jishengella endophytica] | 53.74% |
| 74 | WP_122184786.1 | prenyltransferase [Streptomyces triticirhizae] | 53.69% |
| 75 | WP_143644462.1 | prenyltransferase [Streptomyces sp. IB201691-2A2] | 53.25% |
| 76 | WP_095564968.1 | prenyltransferase [Plantactinospora sp. KBS50] | 53.00% |
| 77 | WP_103783028.1 | prenyltransferase [Streptomyces sp. Ru71] | 52.63% |
| 78 | WP_135330626.1 | prenyltransferase [Streptomyces sp. MZ04] | 52.50% |
| 79 | WP_119294080.1 | prenyltransferase [Streptomyces sp. YIM 130001] | 52.45% |
| 80 | OPG09817.1 | prenyltransferase [Streptomyces sp. GKU 895] | 52.31% |
| 81 | WP_078616106.1 | hypothetical protein [Streptomyces sp. 303MFCol5.2] | 52.30% |
| 82 | WP_132158260.1 | prenyltransferase [Actinomadura sp. 7K507] | 51.99% |
| 83 | WP_079307242.1 | prenyltransferase [Streptomyces sp. GKU 895] | 51.96% |
| 84 | WP_073827930.1 | prenyltransferase [Micromonospora sp. TSRI0369] | 51.21% |
| 85 | WP_040687408.1 | aromatic prenyltransferase [Nocardia vinacea] | 51.19% |
| 86 | WP_150215285.1 | prenyltransferase [Streptomyces venezuelae] | 51.16% |
| 87 | WP_083940321.1 | prenyltransferase [Saccharomonospora saliphila] | 51.04% |
| 88 | WP_104112297.1 | prenyltransferase [Micromonospora chalcea] | 50.87% |
| 89 | EWM63041.1 | hypothetical protein MCBG_00174 [Micromonospora sp. M42] | 50.87% |
| 90 | WP_030499012.1 | aromatic prenyltransferase [Micromonospora purpureochromogenes] | 50.87% |
| 91 | WP_027748955.1 | hypothetical protein [Streptomyces sp. CNH287] | 50.79% |
| 92 | WP_067309254.1 | prenyltransferase [Micromonospora rifamycinica] | 50.71% |
| 93 | WP_064445539.1 | prenyltransferase [Micromonospora sp. NBRC 110037] | 50.52% |
| 94 | WP_063043013.1 | prenyltransferase [Nocardia pseudovaccinii] | 50.17% |
| 95 | WP_117400900.1 | prenyltransferase [Actinomadura sp. LHW52907] | 49.83% |
| 96 | WP_091651703.1 | prenyltransferase [Micromonospora pallida] | 49.83% |
| 97 | WP_084467029.1 | prenyltransferase [Nocardia arthritidis] | 49.49% |
| 98 | WP_145922251.1 | prenyltransferase [Micromonospora sp. HM134] | 49.29% |
| 99 | WP_141955840.1 | prenyltransferase [Actinoallomurus bryophytorum] | 49.17% |
| 100 | WP_063813137.1 | prenyltransferase [Nocardia anaemiae] | 49.15% |
| 101 | WP_083864015.1 | prenyltransferase [Nocardia exalbida] | 49.15% |
| 102 | WP_121435675.1 | prenyltransferase [Actinomadura pelletieri] | 49.13% |
| 103 | WP_116072936.1 | prenyltransferase [Asanoa ferruginea] | 49.13% |
| 104 | WP_020673231.1 | hypothetical protein [Amycolatopsis nigrescens] | 49.11% |
| 105 | WP_104379267.1 | prenyltransferase [Nocardia nova] | 48.99% |
| 106 | WP_037748550.1 | aromatic prenyltransferase [unclassified Streptomyces] | 48.97% |
| 107 | WP_101420943.1 | prenyltransferase [Streptomyces sp. CMB-StM0423] | 48.97% |
| 108 | WP_145782929.1 | prenyltransferase [Streptomyces sp. CNZ287] | 48.92% |
| 109 | AXO35195.1 | hypothetical protein MicB006_2914 [Micromonospora sp. B006] | 48.76% |
| 110 | WP_049572089.1 | hypothetical protein [Streptomyces sp. SBT349] | 48.67% |
| 111 | WP_145899200.1 | prenyltransferase [Streptomyces sp. CNZ289] | 48.62% |
| 112 | WP_047016268.1 | aromatic prenyltransferase [Streptomyces sp. CNQ-509] | 48.56% |
| 113 | WP_063619318.1 | prenyltransferase [Streptomyces sp. CNH099] | 48.56% |
| 114 | WP_099926184.1 | hypothetical protein [Streptomyces sp. 70] | 48.51% |
| 115 | WP_084499306.1 | prenyltransferase [Nocardia gamkensis] | 48.46% |
| 116 | WP_027752515.1 | aromatic prenyltransferase [Streptomyces sp. CNH099] | 48.44% |
| 117 | WP_018839517.1 | hypothetical protein [Streptomyces sp. CNQ766] | 48.40% |
| 118 | WP_104366678.1 | prenyltransferase [Nocardia nova] | 48.32% |
| 119 | WP_100301149.1 | prenyltransferase [Streptomyces sp. CNZ306] | 48.28% |
| 120 | WP_121433960.1 | prenyltransferase [Actinomadura pelletieri] | 48.21% |
| 121 | WP_063015362.1 | hypothetical protein [Corynebacteriales] | 47.99% |
| 122 | WP_073502277.1 | prenyltransferase [Streptomyces paucisporeus] | 47.95% |
| 123 | WP_063790015.1 | prenyltransferase [Streptomyces sp. MMG1121] | 47.95% |
| 124 | WP_137233316.1 | hypothetical protein [Streptomyces sp. BPSDS2] | 47.88% |
| 125 | WP_065481562.1 | hypothetical protein [Streptomyces sp. PTY08712] | 47.76% |
| 126 | WP_145815899.1 | prenyltransferase [Micromonospora sagamiensis] | 47.75% |
| 127 | WP_150521200.1 | cupin domain-containing protein [Streptomyces subrutilus] | 47.74% |
| 128 | WP_039778124.1 | aromatic prenyltransferase [Nocardia cerradoensis] | 47.65% |
| 129 | WP_030517845.1 | hypothetical protein [Nocardia sp. NRRL WC-3656] | 47.65% |
| 130 | WP_027740846.1 | aromatic prenyltransferase [Streptomyces sp. CNT371] | 47.59% |
| 131 | WP_125503295.1 | prenyltransferase [unclassified Streptomyces] | 47.57% |
| 132 | WP_100203158.1 | prenyltransferase [Streptomyces carminius] | 47.57% |
| 133 | WP_101424043.1 | prenyltransferase [Streptomyces sp. CMB-StM0423] | 47.48% |
| 134 | WP_051956317.1 | prenyltransferase [Streptomyces atratus] | 47.33% |
| 135 | WP_063066486.1 | hypothetical protein [Nocardia violaceofusca] | 47.32% |
| 136 | WP_063007124.1 | hypothetical protein [Nocardia kruczakiae] | 47.32% |
| 137 | WP_104364109.1 | prenyltransferase [Nocardia nova] | 47.32% |
| 138 | SUA47165.1 | Aromatic prenyltransferase Orf2 [Nocardia africana] | 47.32% |
| 139 | WP_117399009.1 | prenyltransferase [Actinomadura sp. LHW52907] | 47.28% |
| 140 | WP_049575632.1 | aromatic prenyltransferase [Streptomyces sp. SBT349] | 47.19% |
| 141 | WP_131544751.1 | hypothetical protein [Streptomyces sp. IBTA2] | 47.19% |
| 142 | WP_036499104.1 | hypothetical protein [Nocardia aobensis] | 47.18% |
| 143 | WP_143644477.1 | prenyltransferase [Streptomyces sp. IB201691-2A2] | 47.16% |
| 144 | WP_083880814.1 | prenyltransferase [Nocardia araoensis] | 47.10% |
| 145 | WP_084489194.1 | prenyltransferase [Nocardia niwae] | 47.10% |
| 146 | WP_125619124.1 | hypothetical protein [Streptomyces sp. WAC04770] | 47.06% |
| 147 | WP_063920851.1 | hypothetical protein [Nocardia mikamii] | 46.98% |
| 148 | WP_051866258.1 | prenyltransferase [Streptomyces griseus] | 46.98% |
| 149 | WP_053560640.1 | hypothetical protein [Streptomyces sp. CFMR 7] | 46.86% |
| 150 | WP_106954687.1 | prenyltransferase [Nocardia sp. MDA0666] | 46.64% |
| 151 | XP_025437791.1 | hypothetical protein BO95DRAFT_456766 [Aspergillus brunneoviolaceus CBS 621.78] | 46.46% |
| 152 | WP_084823458.1 | prenyltransferase [Nocardia beijingensis] | 46.42% |
| 153 | WP_097275149.1 | prenyltransferase [Streptomyces sp. TLI_55] | 46.40% |
| 154 | RBL81021.1 | hypothetical protein DDE05_47290 [Streptomyces cavourensis] | 46.34% |
| 155 | WP_073499477.1 | prenyltransferase [Streptomyces paucisporeus] | 46.26% |
| 156 | WP_031521063.1 | aromatic prenyltransferase [Streptomyces sp. NRRL F-5123] | 46.24% |
| 157 | WP_082412569.1 | hypothetical protein [Actinobacteria bacterium OV320] | 46.23% |
| 158 | TCR75458.1 | aromatic prenyltransferase Orf2 [Streptomyces sp. BK042] | 46.18% |
| 159 | SDJ65986.1 | Aromatic prenyltransferase Orf2 [Actinopolyspora mzabensis] | 46.15% |
| 160 | XP_025503118.1 | hypothetical protein BO66DRAFT_325070 [Aspergillus aculeatinus CBS 121060] | 46.13% |
| 161 | WP_040717943.1 | hypothetical protein [Nocardia veterana] | 46.10% |
| 162 | WP_091459402.1 | prenyltransferase [Micromonospora inyonensis] | 46.08% |
| 163 | WP_141973439.1 | prenyltransferase [Actinomadura hallensis] | 45.58% |
| 164 | WP_079073831.1 | hypothetical protein [Streptomyces sp. Root1310] | 45.55% |
| 165 | WP_078890532.1 | hypothetical protein [unclassified Streptomyces] | 44.52% |
| 166 | XP_001589743.1 | hypothetical protein SS1G_09465 [Sclerotinia sclerotiorum 1980 UF-70] | 44.37% |
| 167 | WP_101421570.1 | hypothetical protein [Streptomyces sp. CMB-StM0423] | 44.26% |
| 168 | WP_147874947.1 | hypothetical protein [Streptomyces sp. IB2014 016-6] | 44.22% |
| 169 | PQE07084.1 | Aromatic prenyltransferase protein [Rutstroemia sp. NJR-2017a BVV2] | 44.11% |
| 170 | WP_078616079.1 | hypothetical protein [Streptomyces sp. 303MFCol5.2] | 44.00% |
| 171 | WP_078899560.1 | hypothetical protein [Streptomyces] | 43.88% |
| 172 | WP_078074323.1 | hypothetical protein [Streptomyces niveus] | 43.88% |
| 173 | WP_135330628.1 | hypothetical protein [Streptomyces sp. MZ04] | 43.58% |
| 174 | WP_063826174.1 | hypothetical protein [Streptomyces antibioticus] | 43.54% |
| 175 | WP_117358562.1 | hypothetical protein [Actinomadura sp. NEAU-G17] | 43.45% |
| 176 | PVH76557.1 | hypothetical protein DL98DRAFT_535616 [Cadophoa sp. DSE1049] | 43.45% |
| 177 | WP_047018064.1 | hypothetical protein [Streptomyces sp. CNQ-509] | 43.39% |
| 178 | WP_145784670.1 | hypothetical protein [Streptomyces sp. CNZ287] | 43.39% |
| 179 | AQU65798.1 | hypothetical protein BBN63_05610 [Streptomyces niveus] | 43.33% |
| 180 | MWA08000.1 | hypothetical protein [Streptomyces sp. BA2] | 43.23% |
| 181 | CDH35382.1 | aromatic prenyltransferase [Streptomyces iakyrus] | 43.20% |
| 182 | WP_069463857.1 | hypothetical protein [Streptomyces rubidus] | 43.15% |
| 183 | WP_066951135.1 | hypothetical protein [Microtetraspora fusca] | 43.10% |
| 184 | KJK34409.1 | hypothetical protein UK15_36180 [Streptomyces variegatus] | 43.07% |
| 185 | TVY84701.1 | 4-hydroxyphenylpyruvate 3-dimethylallyltransferase [Lachnellula suecica] | 43.00% |
| 186 | TGO19921.1 | hypothetical protein BTUL_0002g01700 [Botrytis tulipae] | 43.00% |
| 187 | WP_031138137.1 | hypothetical protein [Streptomyces xanthophaeus] | 42.71% |
| 188 | KAA8569914.1 | hypothetical protein EYC84_002254 [Monilinia fructicola] | 42.66% |
| 189 | XP_001560463.1 | hypothetical protein BCIN_06g02600 [Botrytis cinerea B05.10] | 42.66% |
| 190 | WP_063619313.1 | hypothetical protein [Streptomyces sp. CNH099] | 42.57% |
| 191 | WP_133260072.1 | hypothetical protein [Streptacidiphilus pinicola] | 42.52% |
| 192 | KKY13521.1 | hypothetical protein UCRPC4_g06967 [Phaeomoniella chlamydospora] | 42.41% |
| 193 | WP_018840564.1 | hypothetical protein [unclassified Streptomyces] | 42.33% |
| 194 | TGO66701.1 | hypothetical protein BOTNAR_0056g00400 [Botryotinia narcissicola] | 42.32% |
| 195 | WP_078564382.1 | hypothetical protein [Streptomyces sp. CNQ329] | 42.32% |
| 196 | AFI64508.1 | Wt3.9 [Streptomyces sp. WT3] | 42.27% |
| 197 | CCD48995.1 | similar to gi\|310689659\|pdb\|2XM7\|A Chain A [Botrytis cinerea T4] | 42.22% |
| 198 | WP_148356240.1 | hypothetical protein [Actinomadura syzygii] | 42.21% |
| 199 | WP_143644478.1 | hypothetical protein [Streptomyces sp. IB201691-2A2] | 42.16% |
| 200 | WP_126635119.1 | hypothetical protein [Streptomyces hyalinus] | 42.16% |
| 201 | KUN17777.1 | hypothetical protein AQJ23_40535 [Streptomyces antibioticus] | 42.05% |
| 202 | WP_018844883.1 | hypothetical protein [unclassified Streptomyces] | 42.00% |
| 203 | WP_103786661.1 | hypothetical protein [Streptomyces sp. Ru71] | 41.98% |
| 204 | TGO79002.1 | hypothetical protein BELL_0046g00010 [Botrytis elliptica] | 41.98% |
| 205 | TGO61486.1 | hypothetical protein BCON_0027g00760 [Botryotinia convoluta] | 41.98% |
| 206 | WP_078904276.1 | hypothetical protein [Streptomyces xanthophaeus] | 41.98% |
| 207 | OTA08152.1 | hypothetical protein A9Z42_0091200 [Trichoderma parareesei] | 41.89% |
| 208 | WP_109499726.1 | hypothetical protein [Streptomyces sp. Act143] | 41.86% |
| 209 | RAG82183.1 | hypothetical protein DN069_28980 [Streptacidiphilus pinicola] | 41.84% |
| 210 | GAO81753.1 | hypothetical protein AUD_0713 [Aspergillus udagawae] | 41.84% |
| 211 | WP_078982086.1 | hypothetical protein [Streptomyces scabrisporus] | 41.81% |
| 212 | THV47184.1 | hypothetical protein BGAL_0329g00100 [Botrytis galanthina] | 41.78% |
| 213 | WP_100302114.1 | hypothetical protein [Streptomyces sp. CNZ306] | 41.67% |
| 214 | WP_037751911.1 | hypothetical protein [Streptomyces sp. CNQ-525] | 41.67% |
| 215 | WP_103552883.1 | hypothetical protein [Streptomyces populi] | 41.61% |
| 216 | KQX72267.1 | hypothetical protein ASD48_39950 [Streptomyces sp. Root1310] | 41.61% |
| 217 | WP_053666646.1 | hypothetical protein [Streptomyces sp. MMG1121] | 41.58% |
| 218 | SKA30833.1 | Aromatic prenyltransferase Orf2 [Marinactinospora thermotolerans DSM 45154] | 41.58% |
| 219 | WP_078763101.1 | hypothetical protein [Marinactinospora thermotolerans] | 41.58% |
| 220 | WP_130454911.1 | hypothetical protein [Streptomyces sp. CNZ288] | 41.33% |
| 221 | WP_145901163.1 | hypothetical protein [Streptomyces sp. CNZ289] | 41.33% |
| 222 | WP_027745939.1 | hypothetical protein [Streptomyces sp. CNT371] | 41.33% |
| 223 | WP_055569113.1 | hypothetical protein [Streptomyces atriruber] | 41.31% |
| 224 | KAB8298687.1 | hypothetical protein EYC80_000864 [Monilinia laxa] | 41.30% |
| 225 | WP_146478483.1 | hypothetical protein [Streptomyces sp. SSL-25] | 41.16% |
| 226 | WP_122199365.1 | hypothetical protein [Actinomadura sp. NEAU-Ht49] | 41.14% |
| 227 | WP_150213418.1 | hypothetical protein [Streptomyces venezuelae] | 41.10% |
| 228 | TG083018.1 | hypothetical protein BPOR_0718g00070 [Botrytis porri] | 40.96% |
| 229 | WP_143023025.1 | hypothetical protein [Lentzea jiangxiensis] | 40.82% |
| 230 | XP_001210907.1 | predicted protein [Aspergillus terreus NIH2624] | 40.82% |
| 231 | WP_130468244.1 | hypothetical protein [Actinopolyspora sp. DSM 45956] | 40.78% |
| 232 | WP_049571866.1 | hypothetical protein [Streptomyces sp. SBT349] | 40.67% |
| 233 | TG041762.1 | hypothetical protein BHYA_0017g00730 [Botrytis hyacinthi] | 40.61% |
| 234 | WP_084510537.1 | hypothetical protein [Nocardia lijiangensis] | 40.33% |
| 235 | TGO19972.1 | hypothetical protein BPAE_0327g00150 [Botrytis paeoniae] | 40.27% |
| 236 | AGS49802.1 | hypothetical protein [uncultured bacterium esnapd16.1] | 40.21% |
| 237 | WP_142270708.1 | hypothetical protein [Streptomyces sp. SLBN-115] | 40.13% |
| 238 | WP_150163951.1 | hypothetical protein [Streptomyces venezuelae] | 40.07% |
| 239 | WP_150181842.1 | hypothetical protein [Streptomyces venezuelae] | 40.00% |
| 240 | WP_078627940.1 | hypothetical protein [Streptomyces sp. CNH099] | 40.00% |
| 241 | KOV57748.1 | hypothetical protein ADK64_38125 [Streptomyces sp. MMG1121] | 40.00% |
| 242 | XP_024752719.1 | hypothetical protein BBK36DRAFT_1111187 [Trichoderma citrinoviride] | 39.93% |
| 243 | WP_101420456.1 | hypothetical protein [Streptomyces sp. CMB-StM0423] | 39.93% |
| 244 | WP_091373094.1 | hypothetical protein [Alloactinosynnema album] | 39.93% |
| 245 | TEY79915.1 | hypothetical protein BOTCAL_0040g00320 [Botryotinia calthae] | 39.87% |
| 246 | MWA09138.1 | prenyltransferase [Streptomyces sp. BA2] | 39.86% |
| 247 | XP_020123439.1 | hypothetical protein UA08_01803 [Talaromyces atroroseus] | 39.67% |
| 248 | XP_002847323.1 | NovQ [Microsporum canis CBS 113480] | 39.65% |
| 249 | WP_078627401.1 | hypothetical protein [Streptomyces sp. CNH099] | 39.60% |
| 250 | WP_043384953.1 | hypothetical protein [Streptomyces luteus] | 39.54% |
| 251 | WP_104481745.1 | cupin domain-containing protein [Actinokineospora auranticolor] | 39.40% |
| 252 | TQL97062.1 | aromatic prenyltransferase Orf2 [Actinoallomurus bryophytorum] | 39.27% |
| 253 | WP_157429804.1 | hypothetical protein [Actinomadura oligospora] | 39.25% |
| 254 | WP_146478483.1 | hypothetical protein [Streptomyces sp. SSL-25] | 39.19% |
| 255 | WP_141955833.1 | hypothetical protein [Actinoallomurus bryophytorum] | 39.14% |
| 256 | WP_141955837.1 | hypothetical protein [Actinoallomurus bryophytorum] | 39.02% |
| 257 | PMD17248.1 | hypothetical protein NA56DRAFT_648701 [Pezoloma ericae] | 38.75% |
| 258 | WP_063784117.1 | hypothetical protein [Streptomyces sp. SBT349] | 38.56% |
| 259 | WP_084496655.1 | hypothetical protein [Nocardia amamiensis] | 38.51% |
| 260 | WP_078899998.1 | hypothetical protein [Streptomyces sp. SBT349] | 38.19% |
| 261 | WP_092625293.1 | hypothetical protein [Actinopolyspora mzabensis] | 38.02% |
| 262 | CAL34106.1 | putative prenyltransferase [Streptomyces cinnamonensis] | 37.99% |
| 263 | WP_078560068.1 | hypothetical protein [Streptomyces sp. CNT371] | 37.67% |
| 264 | TCO55794.1 | aromatic prenyltransferase Orf2 [Actinocrispum wychmicini] | 37.65% |
| 265 | KJZ73749.1 | hypothetical protein HIM_06867 [Hirsutella minnesotensis 3608] | 37.63% |
| 266 | CEL03594.1 | hypothetical protein ASPCAL04746 [Aspergillus calidoustus] | 37.50% |
| 267 | OOQ89743.1 | hypothetical protein PEBR_07177 [Penicillium brasilianum] | 37.41% |
| 268 | PYH97268.1 | hypothetical protein BO71DRAFT_468204 [Aspergillus ellipticus CBS 707.79] | 37.41% |
| 269 | KAE9377664.1 | hypothetical protein N431DRAFT_527667 [Chalara longipes BDJ] | 37.37% |
| 270 | KUL90715.1 | hypothetical protein ZTR_00003 [Talaromyces verruculosus] | 37.16% |
| 271 | GAM39384.1 | hypothetical protein TCE0_034r10891 [Talaromyces cellulolyticus] | 36.64% |
| 272 | PCH00031.1 | hypothetical protein PENOC_055060 [Penicillium sp. ‘occitanis'] | 36.64% |
| 273 | WP_069462248.1 | hypothetical protein [Streptomyces rubidus] | 36.51% |
| 274 | PQE21594.1 | 4-hydroxyphenylpyruvate 3-DMAPP transferase protein [Rutstroemia sp. NJR-2017a] | 36.46% |
| 275 | OQD84936.1 | hypothetical protein PENANT_c011G06136 [Penicillium antarcticum] | 36.15% |
| 276 | PQE13805.1 | Chain A like protein [Rutstroemia sp. NJR-2017a BBW] | 36.11% |
| 277 | RAO64565.1 | hypothetical protein BHQ10_000577 [Talaromyces amestolkiae] | 35.96% |
| 278 | CRG83396.1 | hypothetical protein PISL3812_00747 [Talaromyces islandicus] | 35.91% |
| 279 | KAA8647038.1 | putative secondary metabolism biosynthetic enzyme [Aspergillus tanneri] | 35.89% |
| 280 | XP_018062656.1 | hypothetical protein LY89DRAFT_339943 [Phialocephala scopiformis] | 35.62% |
| 281 | KIM97227.1 | hypothetical protein OIDMADRAFT_20473 [Oidiodendron maius Zn] | 35.52% |
| 282 | PQE15082.1 | Aromatic prenyltransferase protein [Rutstroemia sp. NJR-2017a BVV2] | 35.02% |
| 283 | KJX93611.1 | Chain A like protein [Zymoseptoria brevis] | 34.71% |
| 284 | KFX50527.1 | Aromatic prenyltransferase NovQ [Talaromyces marneffei PM1] | 34.65% |
| 285 | XP_024705102.1 | hypothetical protein P170DRAFT_358697 [Aspergillus steynii IBT 23096] | 33.67% |
| 286 | KIM92739.1 | hypothetical protein OIDMADRAFT_139131 [Oidiodendron maius Zn] | 33.57% |
| 287 | PVH96444.1 | hypothetical protein DM02DRAFT_569563 [Periconia macrospinosa] | 33.33% |
| 288 | MAR13547.1 | hypothetical protein [Blastopirellula sp.] | 32.75% |
| 289 | RDW66932.1 | hypothetical protein BP5796_09681 [Coleophoma crateriformis] | 32.00% |
| 290 | RDW62099.1 | hypothetical protein BP6252_11532 [Coleophoma cylindrospora] | 30.87% |
| 291 | POS72278.1 | hypothetical protein DHEL01_v209327 [Diaporthe helianthi] | 27.36% |
| 292 | ROW05052.1 | hypothetical protein VSDG_00518 [Valsa sordida] | 27.36% |
| 293 | ROV99468.1 | hypothetical protein VMCG_06385 [Valsa malicola] | 26.71% |
| 294 | KKY34764.1 | hypothetical protein UCDDA912_g05266 [Diaporthe ampelina] | 26.69% |
| 295 | KUI68523.1 | 4-hydroxyphenylpyruvate 3-dimethylallyltransferase [Valsa mali] | 25.26% |

Also described herein are polypeptides encoding enzymes that are typically O-methyltransferases. These polypeptides are typically derived from plants and find use in converting phenolic compounds into compounds with medicinal activity and/or precursors to such compounds. For example, described herein are polypeptides comprising one or more of the following sequences, variants thereof, or fragments of the polypeptides or variants. In the following sequences, amino acid sequences and GenBank accession numbers of O-methyltransferases from Cannabis sativa are shown.

>CsOMT1 [PK03555]
SEQ ID NO: 7
MEADGEDAVLRGQVEIWKYMLSFADSMALKCAVELQLADIIHSHTSPITLSQIASAIPGATSPDLSCLARIMRLLVR
RRIFTQHQKPKSDGEEEEALYGPTHSSRWLLTKINDHDQLTLAPMILMENDPRLMAPWHCFSRCVKEGGVAFKKAHN
GQSIWEFGAENPEINKLENDAMECTAKVVMKAILSHYSDGGFSDIKSMVDVGGGTGGSISEIVRSYPHIKGINEDLP
HVIATAPPYSGVSHVGGDMERSVPTADAIFMKWILHDWSDEDCVKILKNCRKAIAEESGKVIIVESVLEEESNNNNN
NNEVFGDTALMLDLVMVAHTTGGKERTQKQWKTILEQGGFPRYNFIKIKALPSIIEAYPN
>CSOMT2 [PK03696]
SEQ ID NO: 8
SELLFQAQTHLYNHTLSYISSMCLKCAIELGIPDIINNHGQPHIPLPQLVSSLRLPPTKTDILRRLMRPLVHFGYFT
TTKVVINSQNKEEEEEEVDAYGLTSSSKLLFVNNNGNNKIPSMSTIVCLQLDQAFMTPWHSLGNWLRKDEATTLFES
AHEMSFWEYTSKNTKFGHLFNEAMADDSKLMLKLVIEDVKPVFEGLTSLVDVGGGTGEVCKILTQVFPHLKCSVLEL
SHVVANLPNAQNLKFIEGDMFQAIPPANAVLLKWILHNWSDEECVTILKKCREAIASNEGGKVVIIDVVINSKKDEH
EVNEAKISFDLMMMVLENGRERSQKEWENLFFKAGFTRYKITPIFGLESLIEVFP
>CSOMT3 [PK04621]
SEQ ID NO: 9
MASAVKGAILAIDNEASFQDQAEIWKYMLNFGDSMALKCAVELRLPDILHSADGPMTLSEIAAAIPNAPSPQASHLE
RVLRLLVRRKIFTSEEDKVSGETLYGPTKLSSWLLHEPAPTSDSDASIMTLAPMLIMENHPWHVDPWHKLSEFIREG
GLAPFEKEHGYMFDFAAKNPLYNKLLNNAMACTARITIRTLLSHCGDDLENGVGSVVDVGGGTGRFISEIVKSYPHI
KGINFDRSHVISTAPAYPGVTHIGGDMFQEVPSADAVVTKWVLHDWGDELCVKLLKNCRKAIPEKGGKVIIVDIVVE
ADGEGLEDDTGVVEDVLMLAHNTGGKERTEKEWKSLLDQSGFPRYKITKIPALQSVIEAYPN
>CSOMT4 [PK05994]
SEQ ID NO: 10
MMSSINDNTITTTQQLSLGYVNLYKHMLSYASSMALKSAVELGIPDIIFNKGKAQTISLHELVSALQIPPSRTNFLR
RVMRVLVHSGIFTNEKGYNDDKEEEEVYGLTPSSKLLLTNGNNSEVPSVGPFVLSVLEPVTVTAFHLIGNWLKNEDS
PATPFHLANDDGLGLYEYWGKNTDGFGDRLNEGMESDSGTLKFVLKNFKSTFEGITTLVDVGGNTGTMCKMLIEAFP
HLKCSVFDLPYVVEANSHNNTENLKFIEGDMFQTIPEADAILFKLVLCGCSDDESIKILKNCREAISRNGKGKVLII
ENNVINSEKDELLELEAKLYFDMLLLASVTGRERSKKDWENIFFQAGFTHYEITPMFGLEALIEVEP
>CSOMT5 [PK07724]
SEQ ID NO: 11
MDALSRDQAEIFEHMESFVDSMALKCAVELRIADIIHSQDCPISLTQIASKIITNSHNSPMISSSPDNNTMLYLNRI
MTSLVRKKIFTAQYDHDQNNNQTVLYYGVTSKSRWLLRDAKPSLAPLILMENHPIQMAPWHYFSHIIKDQEGSATAY
EKAHGCGIFELASVNGELNKIFNDGMACLGEMVMGAILPAYDVFGCMGSLVDVGGGIGGDLAEIVKSHPHIKGINED
LPHVTATAPESNGVTHVAGNMFESVPSADAIFIKWVLHDWCDEECVKILRNCRKAIPEKNGKVIIVEIVLKDSSQNK
ENDDVEDETRMIFDMVMMAHTCRGKERTEFEWKKLLEEAGFPRYKITKIPAIPSIIEAYPF
>CSOMT6 [PK08183]
SEQ ID NO: 12
MAPTQISEELEASLFAMQLAGTSSILPMVLKTALELDLLEIIAMAGPNAFLSPSDIAAQLPTNNPNASMMLDRMLRL
LASYNVLTYLLRDKVTSDGKVLVERLYGLAPLSKELTKNEDGASIAPLCLMVQDKVEMESWYHLKDAILDGGIAFDK
AHGMPAFKYNQIDKRENKIFNKGMFDHSSITMKKILETYKGFEGLNSMVDVGGGSGAVLSMIVTKYPSIKGINFDLH
HVIEDAPPFPGVAHVGGDMFVSVPKGDAIFIKWICHDWSDEDCLKLLKNCYDAVPHHGKVIVAEFILSVAPDSSLAA
KCTAHSDMIMLVDHGGKERTQKEFEELAKAAGFKGFKVVCNAFNTYIMEFLKTN
>CSOMT7 [PK08793]
SEQ ID NO: 13
MAVETHKDELIWIPKEDEEERARVDIYKYIFGFVEMAVVKCAIELGIADAIESHGRPMSLLELSSALGCAAPALHRI
MRFLTNRKLFKEIRINENVQDSEQPSLYAQTALSRLILRSGEKSMATFVLMESSPPMLAPWHGLSARVKTEVDDSSA
PSPFEVANGKDVWSYAAANPGHSQLINEAMACNARVTVAAILDGCLDVEDGIGTIVDVGGGNGTALRMLVRACPWIR
GINFDLPHVVSVALKSEGVEHVGGDMFKFVPKADAAFLMSVLHDWEDDECIQILKKCREAIPGDKGKVIMVECVIEE
NNNNVEEKHEELELKDVGLFLDMVMIAHTNKGKERTLEEWAYVLAQAGENRYNIRAINAIYSIIEAFPN
>CSOMT8 [PK10317]
SEQ ID NO: 14
MAKMQEGGDHHDELTCRAKEEDEQEEERARIDIYKYVEGFVEMAVVKCAIELGIADTIECHGRPMSLKELSSALGCT
PHNIHRIMRELVNRRIFKEIKNDIVNDEGAGTLYVQTSLSRLLIKSGERSMASFVLMESSKPMLAPWHCLSSRLKAE
VIDNSLTPFEEANGQDLWSYTAANPEHSQLLNEAMACNARVTVAAILDSCLEVEDGVGSIVDVGGGNGTAMQLLVKG
CPWIKEGILFDLPHVVSVALKSDRVVHVGGDMEDSVPKADAAFLMWVLHDWEDKDCIQILKNCREAISEKGKVIIVE
SVIENNKEQNNGMKKDELEFKDVGLFLDMVMMAHTNKGKERTLDQWVYVLHQAGFTRYNVRSIKGAISSLIEAFPI
>CSOMT9 [PK10819]
SEQ ID NO: 15
MEKLKSFSYLNNNIDLAMNEENNTKLLGAQAHIWNQIENFINSMCLKCAVELGIPDIINNYGKPMTISQLTLALSIN
KNKFHCLYRLMRLLTHSGFFALEKVEIEGEKEEEGYVITEASKLLLKDNPMSVTPFLLLVLNPILTKSFDVLDTWFQ
NDSPTPFDTANGRTFWDYGSHEPKLVQLENDGMASDARLVTSVVIEKCKGVFEGVERLVDVGGGTGTVAKSIATAFP
QIECSVLDLPHVVADLEDENNLKFIGGDMFVEIPTADVVLLKWILHDWNDEESVKILKNCREAVYKSKKKSGKLIII
DMNIKNDNNENSFETQLFFDMLMMALVSGRERNEKEWSKLFKDAGFSRYKITPILGLRFVIEVYP
>CSOMT10 [PK11500]
SEQ ID NO: 16
MDGIQEGDHHDELTLRLNEKEEEERARIDIYKYVEGFVEMAVVKCAIELGIADTIESHGRPISLLDLSSALSCNPHN
LHRIMRFLVNRRIFKEIKNDTVNDKGCLYVQTSLSRLLIKSGERSMASFVLMESSNPMLAPWHGLSARVKAEATDAL
TPFEAANGVDVWSYAAANPDHSQLINEAMACNARVTVAAILNGCLDVFDGVGSIVDVGGGNGTTLQLLVKGCPWINQ
GINFDLPHVVSVALKSDGVVHVGGDMEDSVPKADAAFLMWVLHDWGDQECIQILKKCKEAIPEKGKVIIVESVIENN
KLEENVMKKELELKDVGLFLDMTMMAHTNKGKERSLDEWVYVLHQAGFTRYNVRSIDGAVSSVIEAFPA
>CSOMT11 [PK12774]
SEQ ID NO: 17
MGSISENTTTLELSQGYVNVYKHMLSYASSMALKCAIQLHIPDIIFTKGKDQTITLPELASALQIPPSRISCLRRVM
RVLVHSGIFSNKNQHDDDKTEEEEVYGLTSSSKILLTGGNNNGVPSVGGYVLAVLEPVTVTAFHLIGSWLKKESPRT
PFHLANDEGLSLYEYWGKNIDGFGDRLDEGMESDSGVLKFVLKDLKSSFEDITTLVDVGGNTGSMCMMLIEEFPHLK
CTVEDLPYAIEANSHNSTSNLKFVEGDMFQSTFPEADAFLLKSVLSGCSDEECVKILKNCGEAISRNGGGKVMVIDN
NVINTKNDEAAELEAKLYFDMLFMTALTGRERTKKDYENIFYQAGFTRIKITRMFGLKSLIEGEL
>CSOMT12 [PK13022]
SEQ ID NO: 18
MTTPTQMSEELEANYLFAMKLASATVLPMVLKTALELGLLEIIVMAGPGAFLSPSNIVAQLPTKNPNAPVMLDRMLR
LLASYNVLTYSIRDGERLYGMTPLSKFLTKNEGGLSIAPLCHMDQDKVIIDCWYHMKDAVLDGGIPENKAHGMPIFE
YTQRDQRLNKIVNRAMSTLSTIIMKNILETYNGFKGLNSIVDVGGGTGATLSMIIAKYPSIKGINFDLHHVIQHAPP
LPGVEHVSGDMFVSVPKGDAIFMKRICHDWSDEECLKLLKNCYDALDDDGKVIVEELIVPAAPDSSPSTKNSFHYDI
LMMVNLNGKERTQKEYEQLAMEAGFKAFKIHCIAFNSYIMEFLAKGPKVEWSVRVPPLL
>CSOMT13 [PK15692]
SEQ ID NO: 19
MESSQLRGQELICQLIFSYYNTMALKCAVELRIADIIHSHGKPITISQIASDIQSNSNSKSPINIDNLFRIMRILVR
KGVFIEHHDDDHGDSTISLYGLCDSSRCLLWDFDSSLVPFILLNTHPLMMASSHNFGKSVIGDKGNPFENDQDVWSF
ASNNPIFNKLENDAMISGSHMVLRHVLSTYKDSFNCIKGTMVDVGGGVGQVISEIVKSHRHIKGINFDLPHVIATAP
TYDGVTHVGGDMFESIPSADVVFLKWIIHGWNDDACVKILKNCRKAIGEKKNGKIIIVDMVLDPNSNEIFQETRLAM
DLVMLANSNNGKERTELEWKKLLNEAGFSRYKITKNQNILDIIEAFPF
>CSOMT14 [PK17162]
SEQ ID NO: 20
MGSINENTITTTQELSQGYVNLYKHMLSYASSMALKSAVELGIPDIIFTKGKAQTISLHELVSALQIPPSRTNELRR
VMRVLVHSGIFTNEKGYNDDKEEEEVYGLTPSSKLLLTGGKNNGVPSVGPYVLAVLEPVTVTAFGSIGNWLKKESPT
TPFHLANDEGLSLYEYWGKNTDGFGDRLNEGMESDSGVLKFVLKDFKSVFEGITTLVDVGGNTGLMCKMLLEAFPHL
QCSVYDLAYAVDANSHNNTQNLKFIEGDMFQTVPQADAILFKCVLSGCSDEECTKILKNCRDAISRNGGGKVLIIDN
NVINSKTEDHLAMETLLYEDMLMMTALTGRERTKKDWEKIFFEAGFSSCKVTPMFGVKTLIEVEP
>CSOMT15 [PK19674.1]
SEQ ID NO: 21
MGSELEGTTEVVVDLKRKQEEESFCYAAQLLNTNVLTKSLQTTIELGIFDIIAKAGEGGKLSAREIVAQLPTNNPDA
PMMVDRILRMLASYSVLVCSVVADDQRAYSLNNVSKCFVTNEDGVSLGPLMLLLEDKVESDSWSQLKGAILEGGIPF
NRFHGMNAFEYPALDSRENKVENRAMQSMTTMLAKQTIESYKGFENLKQLVDVGGGLGVTLKEITSTYPHIKGINED
LPHVVQHAPSYPGVEHVGGDMFESVPSGDAIFMKWILHDWSDEQCVKVLKNCYKAIPENGKVIVMEGLLPMLPEASY
GDNIMSKTDVLMMTQNPGGKERSKQEFQALASGAGENGIRFECCVSGFWIMEFFK
>CsOMT16 [PK19674.2]
SEQ ID NO: 22
MAPPSEELANTPQIVNDERKQEEENFAYAAQLVNSSVLSMSLQSAIELGVEDIIAKAGDAAKLSAQDIVAQMPTTNP
DAPRMLDRILRMLASHSVLACSLENEDLRVYCLNDVSKLFVTNEDGVSLGPLMLLQDKVELDSWSQLKGAILEGGIP
FNRVHGMHAFEYPSLDQKFNQVENKAMYNQTTLVLKKILEVYKGFENLEKVVDVGGGLGGTLNQITSKYPHIKGINF
DLPHVVEHAPSYPGVEHVGGDMFESVPTGAIFMKWILHDWSDEHCLKLLKNCYKAIPDNGNVIVMEAILPTIPETNS
ADRCTSQTDVLMMTQNPGGKERSKQEFQALASGAGENGIRFECCVSGEWIMEFFK
>CSOMT17 [PK19674.3]
SEQ ID NO: 23
MMGSDQLEVIVDLKRPKQEESFCYALQLLSTNILIKSLQATVELGIFDIIAKAGEGSKLSAAEIVAQLPTNNPDAVM
MVDRILRMLAGHSVLTCSVVADNPRVYSHNTVSKCFVTDEDGVSLGSLISLLDDKVYSDSWSQLKGAILEGGIPENR
LHGMNSFEYTALDSRENKVENRAMQSMTTMIAKQTIESYKGFENLKQLVDVGGGLGVTLKEITSTYPHIKGINEDLP
HVVQHAPSYPGVEHVGGDMFESVPSGDAIFMKWILHDWSDEQCVKVLKNCYKAIPENGKVIVMEGLLPMLPEASYGD
NIMSKTDVLMMTQNPGGKERSKQEFQALASGAGENGIRFECCVSGFWIMEFFK
>CSOMT18 [PK19674.4]
SEQ ID NO: 24
MAPPSEELANTPQIVNDERKQEEENFAYAAQLVNSSVLSMSLQSAIELGVEDIIAKAGDAAKLSAQDIVAQMPTTNP
DAPRMLDRILRMLASHSVLACSLENEDLRVYCLNDVSKLFVTNEDGVSLGPLMSLLQDKVELDSWSQLKGAILEGGI
PFNRVHGMHAFEYPSLDQKENQVENKAMYNQTTLVLKKILEVYKGFENLEKVVDVGGGLGGTLNQITSKYPHIKGIN
FDLPHVVEHAPSYPGVEHVGGDMFESVPTGDAIFMKWILHDWSDEHCLKLLKNCYKAIPDNGNVIVMEAILPTIPET
NSADRCTSQTDVLMMTQNPGGKERSKKEFLALATGAGFSGIRFECFVCNEMIMEFYK
>CSOMT19 [PK19715]
SEQ ID NO: 25
MEKSRNSSSHVDLVVNEDNNTKLLRAQAHIWNHICKFINSMSLKCAIELGIPDIVNNHGKPMTISQLTLALPINKNK
SHCLYRLMRLLTHSGFFALEKTEIKGEEEEEGYVITEASKLLLKDNPMSVTPLLLVLLDPTLTKPYDVLSTWERNDD
STPFVTTNGMAIWDYYSHEPKLAQSENEAMASDARLVTSVLIEKCKGVFEGVDSLVDVGGGTGTVAKSIATTEPQIQ
CSVLDLPHVVAGLQGEKNLNFIAGDMFVEVPTAQVVLLKWILHDWSDENSVKILKKCKEAITKSGKKIGKVVVIDMI
IENEKGEIDDESYETQLFMDMTMTLVSGRERNEKELSKLFKDAGFSHYKITPILGLRSLIEIYP
>CSOMT20 [PK23308]
SEQ ID NO: 26
MEKLKSFRHLNNNIDLVLNEENSIELLRAQGHIWNQIENFINSMSLKCAIQLGIPDIINNYGKPMTISQLKLALPIN
QKKSSCVYRLMRILTHSNFFALQKVEGREGEEEEEGYVITDASKLLLKDNPMSVTPFLLAMLDPVITKPWDFLSNWE
QNDDPTPFDTANGMTFWDYGSHQPNLARFFNDAMASDARLVTSVVIEKCRWVFEGVESLVDVGGGTGTVATTIATSE
PQIQCSVLDLPHVVADLQGANNLVNFIGGDMFVEVPPAEVVLLKWILHDWNDEESVKILKKCKEAITKNNKKGGKVI
IIDMKVENEKDEDDESYETQLFFDMLMMALVTGKERNEKEWAKLFKDAGESDYKITPILGLRSLIEVYP
>CSOMT21 [PK24150]
SEQ ID NO: 27
MGSTGIETQMTPTQISDEEANLFAMQLASASVLPMVLKAALELDLLEIIAKAGPGAFLSPSDIAQQLPTQNPDAPVM
LDRMLRLLASYNVVTYSLRERETAEEEGKVERLYGLAPVSKYLTKNEDGVSIAPLCLMNQDKVLMESWYHLKDAVLD
GGIPFNKAYGMTAFEYHGTDQRENKIFNRGMSDHSTITMKKILETYKGFEGLNSIVDVGGGTGAVVNMIVSKYPTIK
GINFDLPHVIEDAPPLTGVEHVGGDMFVSVPKGDAIFMKWICHDWSDEHCLKELKNCHAALPEHGKVIVAECILPVA
PDSSLATKSTVHIDVIMLAHNPGGKERTEKEFEALAKGAGEKGFKVHCNAFNTHIMEFLKTI
>CSOMT22 [PK27112]
SEQ ID NO: 28
MNLIMGEGELVSCRELVEAQELIYNCSLSHIKPMSLKCAIELGIPDIIHNHGQPITLSKLISSLPIHPSKAHCIHRL
MRILVHFGFFTTQLLLPQQQEETYSLTLASKLFLKDCPIKATPFFLVQLNPLLLKPWHELSTWLQKGEDDDDHPSTP
FEMANGINFWDGVGNDPMVKYMFTEAMATDSYLMSKVIVEEGKEVFEGLSSLVDVGGGTGIMANAIVEAFTNIKCTV
LDLPYIVADLKGTHNLNFVEGDMFKKIPSANAVLLKWTLHDWNDEEVVVILKKCREAIWSKDKGGKVIVIDMVIDDD
EEPKSSVETQLCFDMLMMVNLTGKERNEKEWENLFLAAGESHYKINPIVGERSLIEVEP
>CSOMT23 [PK27154]
SEQ ID NO: 29
MEKLKSFRHLNNNIDLVLNEENSIELLGAQGHIWNQIFNFINSMSLKCAIQLGIPDIVNNYRKPMTISQLVLALPIN
QKKSPCVYRLMRILIHSGFFALQKVEGGGEGEEEEGYVITDASKLLLKDNPMSVTPFLLSMLDPVMTKPWDELSNWE
QNDDPTPFDTANGMTFWDYGSHQPNLARFENDAMASDARLVTSVVIEKCKWVFEGVESLVDVGGGTGTVATSIATNE
PQIQCTVLDLPHVVADLQGGNNLNFVGGDMFVEVPTAEVVLLKWILHDWNDEESVKILKKCKEAIMKSKKKGGKVII
IDMKVENEKDEDDESYETQLFFDMLMMTLLTGKERNEKEWAKLFKDAGESDYKITPILGLRSVIEVYP
>CSOMT24 [PK29262]
SEQ ID NO: 30
MQKGQKGCQINQIPMSIERNNVEEDESFFYAVELRSSVVLPMSLYATIELGVFEILAKAGDGAKLSSSDIASHLPTE
NPDAPMMLDRILTLLASHSVLDCVVVGEGSSMRKLYSLSPVSKHELPKEDGVSSHALMKLGLDKVSLESWFELKNAV
LEGGTSFKRAHGMNVFEYGKSDSRFGEVENSAMYNQAKIVTKKIIESYKGFENNIKTLVDVGGGFGVTVSLIVSKYP
QIKAINFDLPHVIKNAPTYPGVEHVGGDMFEKIPNGDAIFMKWILHDWNDEDCVKILKKCYEAIPSNGKVIVVDMVV
PIMAETTHKAKSIFQLDLVMLSQNPGGKERNQHEFQAIANAAGESTINFACSIENVKVIEFIK

The variants of the polypeptides described herein may have any degree of sequence identity to the polypeptides, provided they retain some degree of native activity, for example prenyltransferase or O-methyltransferase activity. For example, the variants typically have at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 99.5% sequence identity.

Likewise, the fragments of the polypeptides or variants described herein may have any length, provided they retain some degree of native activity, for example, prenyltransferase or O-methyltransferase activity. For example, the fragments may be missing about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 125, about 150, about 175, about 200, or about 250 amino acid residues as compared to the polypeptide in question.

The polypeptides, variants, and fragments described herein may also be fused to other polypeptides and could, therefore comprise additional amino acid residues, such as for example about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 125, about 150, about 175, about 200, about 250, about 300, about 400, about 500, about 600, about 700, about 800, about 900, or about 1000 or more additional amino acids.

The polypeptides described herein may comprise, consist of, or consist essentially of the sequences of SEQ ID NO:1-30, or those represented by the accessions in Table 1, and typically encode an enzyme. Typically, the enzyme prenylates a polyphenol, such as a flavonoid, stilbenoid or bibenzyl in the presence of GPP, DMAPP or FPP. In other aspects, the enzyme adds a methyl radical to a polyphenol, such as a flavonoid, stilbenoid or bibenzyl.

The polypeptides described herein are typically expressed in a host cell or organism, such as a bacterium, an archaeon, a yeast, a protozoon, an alga, a fungus, or a plant, including single cells and cell cultures of any thereof for enzymatically acting on a molecule present in the host cell or organism or its cell culture medium. The polypeptides described herein may instead be used in a cell-free system for acting on a molecule present in the system.

In embodiments, the hosts described herein endogenously express and/or are engineered to express at least one nucleic acid coding for an aromatic prenyltransferase polypeptide that is suitable for prenylating a polyphenol, such as a flavonoid, a stilbenoid or a bibenzyl or variants thereof using geranyl diphosphate (GPP), or dimethylallyl diphosphate (DMAPP), or farnesyl diphosphate (FPP) (or variants of either thereof) to produce a prenylated polyphenol, such as a prenylated flavonoid, a stilbenoid or a bibenzyl, or variants thereof. In other embodiments, the engineered host expresses at least one nucleic acid coding for an O-methyltransferase polypeptide that is suitable for methylating a polyphenol, such as a flavonoid, a stilbenoid or a bibenzyl or variants thereof using a methyl (—CH3) donor molecule such as S-adenosyl methionine to produce a methylated polyphenol, such as a methoxy-flavonoid, methoxy-stilbenoid or methoxy-bibenzyl, or variants thereof.

Host cells described herein can be any cell capable of producing at least one protein described herein and include bacterial, fungal (including yeast), animal, algal, and plant cells. The cells may be prokaryotic or eukaryotic. Typical host cells are bacterial, yeast, algal and plant cells. In a typical embodiment, the plant cell is a seed cell, in particular, a cell in a cotyledon or endosperm of a seed. In one embodiment, the cell is a bacterial cell. An example of a bacterial cell useful as a host cell of the present invention is Escherichia coli, Synechococcus spp. (also known as Synechocystis spp.), for example, Synechococcus elongatus. Examples of algal cells useful as host cells of the present invention include, for example, Chlorella sp., Chlamydomonas sp. (for example, Chlamydomonas reinhardtii), Dunaliella sp., Haematococcus sp., Schizochytrium sp., and Volvox sp.

Further exemplary prokaryotic and eukaryotic host cell species are described in more detail below. However, it will be appreciated that other species not specifically described may be suitable.

For example, a recombinant host can be an Ascomycete. A recombinant host can be of a genus selected from the group consisting of Aspergillus, Candida, Pichia, Saccharomyces, and Zygosaccharomyces. A recombinant host can be a photosynthetic microorganism. A recombinant host can be a cyanobacterium selected from the group consisting of Synechocystis, Synechococcus, Athrospira (Spirulina), Anabaena, Rhodopseudomonas. For example, the organism can be of a genus selected from the group consisting of Chlamydomonas, Dunaliella, Chlorella, Botryococcus, Nannochloropsis, Physcomitrella and Ceratodon.

Thus, it will be understood that the polypeptides described herein can be expressed in a variety of expression host cells e.g., bacteria, yeasts, mammalian cells, plant cells, and algal cells, or cell-free expression systems. In one embodiment, described herein are expression vectors comprising the coding DNA sequence for the polypeptides described herein for the expression and purification of the recombinant polypeptide produced from a protein expression system using host cells selected from, e.g., bacteria, mammalian, insect, yeast, or plant cells. In some embodiments, the nucleic acid can be subcloned into a recombinant expression vector that is appropriate for the expression of fusion polypeptide in bacteria, mammalian, yeast, or plant cells or a cell-free expression system such as the wheat germ cell-free expression system or a rabbit reticulocyte expression system. Examples of expression vectors and host cells are the Pichia expression vectors pPICZα, pPICZ, pFLDα and pFLD (Invitrogen) for expression in P. pastoris and vectors pMETα and pMET for expression in P. methanolica; pYES2/GS and pYD1 (Invitrogen) vectors for expression in yeast S. cerevisiae; pET system vectors (Novagen); pGEX (Promega) for expression in E. coli; pBIN and pCAMBIA vectors for expression in plant cells; the strong CMV promoter-based pcDNA3.1 (Invitrogen) and pCINE0 vectors (Promega) for expression in mammalian cell lines such as CHO, COS, HEK-293, Jurkat, and MCF-7; replication incompetent adenoviral vector vectors pADENO-X™, pAd5F35, pLP-ADENO™-X-CMV (CLONTECH®), pAd/CMV/V5-DEST, pAd-DEST vector (Invitrogen) for adenovirus-mediated gene transfer and expression in mammalian cells; pLenti4A/5-DEST™, pLenti6A/5-DEST™, and pLenti6.2/V5-GW/lacZ (Invitrogen) for lentivirus-mediated gene transfer and expression in mammalian cells; BACpak6 baculovirus (Clontech) and pFASTBAC™ HT (Invitrogen) for the expression in S. frugiperda 9 (Sf9), Sf11, Tn-368 and BTI-TN-5B4-1 insect cell lines.

In some embodiments, cell-free systems can include in vitro enzymatic reactions performed in tubes, columns, chips or any other solid support or surface where the prenyltransferase or O-methyltransferase polypeptide described herein is present in solution or immobilized in a resin or another solid support matrix. A range of reversible physical adsorption and ionic linkages, to irreversible stable covalent bonds exist to produce immobilized enzymes. Such techniques include: (a) physical adsorption (for example with cellulose crystals, sol-gel silica, hydroxyapatite, activated carbon, TiO₂ nanoparticles, polyethersulphone membrane, or Ni-/Co-/Zn-nitrilotriacetic acid-agarose); (b) entrapment (for example with agarose or chitosan); and (c) covalent attachment/cross-linking (for example using polyaniline, polystyrene, polyvinyl alcohol, polypropylene, silica gel, bentonite, magnetic nanoparticles, multi-walled carbon nanotubes, reduced graphene oxide, cellulose-poly(acrylic acid) fibers, graphene oxide-Fe3O4, polyacrylonitrile-multi-walled carbon nanotubes, silica-graphene oxide particles).

Thus, in embodiments, the host cells or cell-free systems described herein are suitable for producing a substantially pure prenylated and/or methylated flavonoid, a prenylated and/or methylated stilbenoid or a prenylated and/or methylated bibenzyl, in the presence of at least an aromatic prenyltransferase or an O-methyltransferase polypeptide, such as those represented by SEQ ID:1-30 or those represented by the accessions in Table 1 (or fragments or variants thereof). In embodiments, the host cells or cell-free systems can include a previous modification step such as a nucleic acid coding for an O-methyltransferase polypeptide that is suitable for methylating luteolin to produce chrysoeriol which can then be prenylated by a polypeptide described herein into a substantially pure Cannflavin, such as Cannflavin A and/or B. It will be understood that an O-methyltransferase may be optional when a Cannflavin precursor such as chrysoeriol is produced and/or available to the host cell, the host cell or cell-free system may also comprise a polypeptide for an O-methyltransferase. Likewise, genes encoding one or more enzymes involved in the upstream production of luteolin, apigenin, or naringenin, such as F3′H, FNS, CHI, CHS, 4CL, C4H, or PAL, may also be expressed in the host cells or present in an in vitro system and/or sources of these precursor molecules may be provided exogenously or endogenously. In embodiments, production of pure cannflavin, such as cannflavin A and/or B can also include combining the methylation of luteolin with a subsequent prenylation of chrysoeriol with GPP or DMAPP achieved by means of an organic chemistry synthesis method.

Also described herein are compositions comprising a prenylated and/or methylated flavonoid, a prenylated and/or methylated stilbenoid, or a prenylated and/or methylated bibenzyl obtainable or obtained by one of the methods as disclosed above, and to the use of said composition as a medicinal agent, such as an anti-inflammatory or anti-cancer agent, for pharmacological purposes and/or cosmetic purposes.

Provided herein are compositions comprising substantially pure prenylated and/or methylated flavonoid (for example cannflavin A or cannflavin B), a prenylated and/or methylated stilbenoid, or a prenylated and/or methylated bibenzyl, which are, for example, at least about 95%, about 96%, about 97%, about 98%, about 99%, about 99.5%, or about 99.9% pure.

The compositions comprising a modified flavonoid, stilbenoid or bibenzyl described herein may be formulated for use by a subject, such as a mammal, including a human. Such compositions may comprise about 0.00001% to about 99% by weight of the active and any range there-in-between. For example, typical doses may comprise from about 0.1 μg to about 100 μg of the molecules described herein per 300 mg dose, such as about 0.5 μg, about 1 μg, about 2 μg, about 3 μg, about 4 μg, about 5 μg, about 6 μg, about 7 μg, about 8 μg, about 9 μg, about 10 μg, about 25 μg, about 50 μg, or about 75 μg per 300 mg dose, such as from about 0.1 μg to about 10 μg, or from about 1 μg to about 5 μg, or from about 1 μg to about 2 μg per 300 mg dose (and all related increments and percentages by weight).

The prenylated and/or methylated molecules described herein may be used in any suitable amount, but are typically provided in doses comprising from about 1 to about 10000 ng/kg, such as from about 1 to about 1000, about 1 to about 500, about 10 to about 250, or about 50 to about 100 ng/kg, such as about 1, about 10, about 25, about 50, about 75, about 100, about 150, about 200, about 250, about 300, or about 500 ng/kg. Similar amounts, higher amounts, or lower amounts could be used for administration.

The prenylated and/or methylated molecules described herein may be administered over a period of hours, days, weeks, or months, depending on several factors, including the severity and type of the inflammation or other condition being treated, whether a recurrence is considered likely, or to prevent the inflammation or other condition, etc. The administration may be constant, e.g., constant infusion over a period of hours, days, weeks, months, etc. Alternatively, the administration may be intermittent, e.g., the molecules may be administered once a day over a period of days, once an hour over a period of hours, or any other such schedule as deemed suitable.

The compositions described herein can be prepared by per se known methods for the preparation of pharmaceutically or cosmetically acceptable compositions which can be administered to subjects, such that an effective quantity of the active substance is combined in a mixture with a pharmaceutically acceptable vehicle. Suitable vehicles are described, for example, in “Handbook of Pharmaceutical Additives” (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456 (the entirety of which is incorporated herein by reference).

Pharmaceutically acceptable carriers are well known to those skilled in the art and include, for example, sterile saline, lactose, sucrose, calcium phosphate, gelatin, dextrin, agar, pectin, peanut oil, olive oil, sesame oil, cannabis oil, and water. Furthermore, the composition may comprise one or more stabilizers such as, for example, carbohydrates including sorbitol, mannitol, starch, sucrose, dextrin and glucose, proteins such as albumin or casein, and buffers like alkaline phosphates.

The prenylated and/or methylated molecules described herein can, in embodiments, be administered for example, by parenteral, intravenous, subcutaneous, intradermal, intramuscular, intracranial, intraorbital, ophthalmic, intraventricular, intracapsular, intraspinal, intracisternal, intraperitoneal, intranasal, intrarectal, intravaginal, aerosol, oral, topical, or transdermal administration. Typically, the compositions of the invention are administered orally or topically directly to the site of inflammation or in a cosmetic oil, lotion, cream, or gel to a desired body location, such as the face.

It is understood by one of skill in the art that the produced molecules described herein can be used in conjunction with known therapies for prevention and/or treatment of inflammation in subjects and/or with compositions for preventing the signs of aging or other cosmetic compositions. Similarly, the produced modified molecules described herein can be combined with one or more other pharmaceutical or natural health products, such as cannabinoids, terpenes, or other natural or synthetic compounds. The produced molecules described herein may, in embodiments, be administered in combination, concurrently or sequentially, with conventional treatments for inflammation, including non-steroidal anti-inflammatory drugs, for example. The prenylated and/or methylated molecules described herein may be formulated together with such conventional treatments when appropriate. Other uses of these prenylated and/or methylated molecules can be found due to their anticipated antiatherosclerotic, anticancer, antiviral, antimicrobial or hepatoprotective activities.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### Methods

Chemicals and reagents. Authentic flavonoid standards for chrysoeriol, apigenin, luteolin, kaempherol and quercetin, dihydroresveratrol, tristin, gigantol, batatasin III, resveratrol, pinosylvin, caffeic acid and dihydrocaffeic acid can be purchased from specialized chemical companies such as Toronto Research Chemicals, Indofine Chemical Company or Sigma-Aldrich. The trans-prenyl diphosphates: dimethyllalyl diphosphate (DMAPP), isopentenyl diphosphate (IPP), and geranyl diphosphate (GPP) can be obtained from Echelon Biosciences. Radiolabeled S-[Methyl-14C] adenosyl-L-methionine (58.0 mCi mmol-1) can be obtained from PerkinElmer.

Expression of recombinant prenyltransferases in E. coli. In typical aspects, open reading frames encoding soluble aromatic prenyltransferases from bacteria or fungi, for example NphB, HypSC, CloQ, NovQ, Fur7, or PpzP are synthesized commercially. This cDNA is amplified by PCR and then inserted into an expression vector system (for example, Novagen's pET28) which introduces an N-terminal 6×His tag to the coding sequence. The construct is then introduced into a bacterial host (for example E. coli BL21-CodonPlus (DE3)-RIPL) cell. Bacterial cells expressing recombinant NphB are cultured for several hours in the presence of IPTG to induce recombinant protein expression. The bacterial cells are collected by centrifugation, re-suspended, and then disrupted by sonication. Crude protein extracts are applied and purified with a Ni²⁺ affinity matrix (for example a HisTrap HP column). Afterwards, the enzyme is eluted with high concentrations (250 to 400 uM) of imidazole and then equilibrated in a suitable buffer. The purified NphB protein can be frozen prior to use.

Enzymatic prenylation reactions. Typically, enzyme reactions can be carried out in assay tubes by mixing purified prenyltransferase enzyme, the flavonoid, stilbenoid or bibenzyl substrate (for example, chrysoeriol, kaempferol, or quercetin), a prenyl donor group (GPP, or DMAPP), MgCl₂, and Tris-HCl buffer and incubated in a 30° C. water-bath overnight. Reactions can be stopped by adding 20% formic acid and then extracted with ethyl acetate. For analysis, usually, reactions are dried under N₂ and then resuspended in methanol before separating them on a HPLC with a reverse-phase column (for example Spherisorb ODS2) and eluted with a linear gradient of methanol:water.

Recombinant protein expression of CsOMTs in E. coli. Open reading frames corresponding to O-methyltransferases from C. sativa are synthesized by commercially. These cDNAs are amplified by PCR using a high fidelity DNA polymerase and cloned and expressed following the method described above for prenyltransferase genes.

O-methyltransferase enzyme assays. Assays for determining O-methyltransferase enzyme activity are performed using purified recombinant protein incubated in a final reaction volume of 100 μL of 1 mM substrate and radiolabeled adenosyl-L-methionine in 50 mM Tris-HCl, pH 7.5, 5 mM MgCl2, and 10% (v/v) glycerol for 30 min at 37° C. The enzymatic products are extracted with four volumes of ethyl acetate and quantified using a scintillation counter. For reaction product identification, assays are scaled up to a final volume of 500 μL containing 50 to 200 μg of recombinant protein, 2 mM of substrate and 2 mM S-adenosyl-L-methionine in the same buffer as above. Enzymatic products are extracted as above, evaporated to dryness under N₂ gas, and resuspended in 100 μL of methanol. Samples are analyzed by HPLC with a Spherisorb ODS2 reverse-phase column and eluted over a 20 min gradient from 45% to 95% methanol with 0.1% formic acid (v/v) followed by 100% methanol for 10 min. The eluted products are detected by absorption at the 210-350 nm range and quantified relative to authentic standards. Mass spectral analysis of the enzymatic products is performed as described below.

Mass spectrometry analysis of enzymatic reaction products. The prenylated or methylated products can be purified by HPLC as described above. Generally, samples are evaporated under nitrogen and then re-suspended in methanol prior to liquid chromatography mass spectrometry (LC-MS) analysis (for example HPLC liquid chromatography interfaced with a Q-TOF mass spectrometer). During such analysis, the mass-to-charge ratio is typically scanned across the m/z range of 100-3000 m/z in an extended dynamic range positive-ion MS mode. Chromatograms can be analyzed by using a software that compares MS patterns from standard libraries or known standards used in the laboratory. Fragmentation patterns of the various parent (molecular) ions obtained using collision energies of 5 to 20 V from the recovered peak products (modified compounds) are usually also compared with fragmentation patterns of standard molecules.

In typical aspects, nuclear magnetic resonance (NMR) spectroscopy, is used as a preeminent technique for determining the structure of the prenylated or methylated compounds that can be obtained (for example for determining the position of the GPP, DMAPP, or methyl group in one of the flavonoid, stilbenoid or bibenzyl rings). After the enzymatic reaction products from the enzymatic assays are resolved by HPLC, the compounds are eluted and subsequently collected. Usually, approximately 0.5 mg of each compound is evaporated to dryness under N₂ gas, resuspended in acetone-d6, and analyzed using ¹H and ¹³C NMR. NMR spectra are collected on a spectrometer (for example a Bruker AVANCE III 600 MHz equipped with a 5 mm TCI cryoprobe).

In an exemplary extraction step, this or a similar in vitro cell-free system described herein can be suitable for producing a substantially pure prenylated flavonoid, a prenylated stilbenoid or a prenylated bibenzyl in the presence of at least an aromatic prenyltransferase described herein. The prenylated product can undergo further purification if necessary depending on the in vitro system selected before being destined to be used in preparations.

### REFERENCES

Akao Y, Maruyama H, Matsumoto K, Ohguchi K, Nishizawa K, Sakamoto T, Araki Y, Mishima S, Nozawa Y (2008) Cell growth inhibitory effect of cinnamic acid derivatives from propolis on human tumor cell lines. Biol Pharm Bull. 26: 1057-1059.
Akinwumi B C, Bordun K M, Anderson H D (2018) Biological Activities of Stilbenoids. Int J Mol Sci. 19: E792.
Bandyukova V A, Avanesov E T (1971) The structure of methoxylated flavonoids. Chem Nat Compounds 7: 250-253.
Barron D, Ibrahim R K (1996) Isoprenylated flavonoids—a survey. Phytochemistry 43: 921-982.
Bernini R, Crisante F, Ginnasi M C (2011) A convenient and safe O-methylation of flavonoids with dimethyl carbonate (DMC). Molecules. 16: 1418-1425.
Bonitz T, Alva V, Saleh O, Lupas A N, Heide L (2011) Evolutionary relationships of microbial aromatic prenyltransferases. PLoS One 6: e27336.
Botta B, Delle Monache G, Menendez P, Boffi A (2005) Novel prenyltransferase enzymes as a tool for flavonoid prenylation. Trends Pharmacol Sci 26: 606-608.
Chen X, Mukwaya E, Wong M S, Zhang Y (2014) A systematic review on biological activities of prenylated flavonoids. Pharm Biol. 52: 655-660.
Kikuchi H et al. (2019) Chemopreventive and anticancer activity of flavonoids and its possibility for clinical use by combining with conventional chemotherapeutic agents. Am J Cancer Res 9: 1517-1535.
Koirala N, Thuan N H, Ghimire G P, Thang D V, Sohng J K (2016) Methylation of flavonoids: Chemical structures, bioactivities, progress and perspectives for biotechnological production. Enzyme Microb Technol. 86:103-116.
Kumano T, Richard S B, Noel J P, Nishiyama M, Kuzuyama T (2008) Chemoenzymatic syntheses of prenylated aromatic small molecules using Streptomyces prenyltransferases with relaxed substrate specificities. Bioorg Med Chem. 16: 8117-8126.
Kumar S, Pandey A K (2013) Chemistry and biological activities of flavonoids: an overview Sci. World J 2013: 162750.
Mishima S, Ono Y, Araki Y, Akao Y, Nozawa Y (2005) Two related cinnamic acid derivatives from Brazilian honey bee propolis, baccharin and drupanin, induce growth inhibition in allografted sarcoma S-180 in mice. Biol. Pharm. Bull. 28: 1025-1030.
Ozaki T, Mishima S, Nishiyama M, Kuzuyama T (2009) NovQ is a prenyltransferase capable of catalyzing the addition of a dimethylallyl group to both phenylpropanoids and flavonoids. J Antibiot 62: 385-392.
Paddon C J, Keasling J D (2014) Semi-synthetic artemisinin: a model for the use of synthetic biology in pharmaceutical development. Nat Rev Microbiol. 12: 355-367.
Rasouli H et al. (2017) Polyphenols and their benefits: A review. Int J Food Properties 20: S1700-S1741.
Thodey K, Galanie S, Smolke C D (2014) A microbial biomanufacturing platform for natural and semisynthetic opioids. Nat Chem Biol. 10: 837-844.
Walle T (2007) Methylation of dietary flavones greatly improves their hepatic metabolic stability and intestinal absorption. Mol Pharm. 4: 826-832.
Xiao J, Muzashvili T S, Georgiev M I (2014) Advances in the biotechnological glycosylation of valuable flavonoids. Biotechnol Adv. 32: 1145-1156.
Xiao K, Zhang H J, Xuan L J, Zhang J, Xu Y M, Bai D L (2008) Stilbenoids: Chemistry and bioactivities. Studies Nat Prod Chem 34: 453-646
Yang X, Jianga Y, Yang J, He J, Sun J, Chen F, Zhang M W, Yang B (2015) Prenylated flavonoids, promising nutraceuticals with impressive biological activities. Trends Food Sc Tech 44: 93-104.

### EXAMPLES

### Example 1

This example describes a general method for using the prenyltransferase NphB from Streptomyces sp. strain CL190 to prenylate chrysoeriol using GPP and to produce cannflavin A. A synthetic cDNA sequence of NphB was sub-cloned in the pET28a vector system (Novagen) which introduced an N-terminal 6×His tag to the coding sequence and then the construct was introduced into E. coli BL21-CodonPlus (DE3)-RIPE cells. Bacterial cells expressing recombinant NphB were cultured in 1 L of LB media at 37° C. to an OD 600 of 0.6. Isopropyl-β-D-thiogalactoside (IPTG) was then added to a final concentration of 1 mM and the cells were incubated at 16° C. for an additional 18 h to induce recombinant protein expression. The bacterial cells were collected by centrifugation, re-suspended in 20 mM Tris-HCl, pH 8.0, 500 mM KCl (Buffer A), and then disrupted by sonication. Crude protein extracts were centrifuged at 12,000×g for 10 min at 4° C. to remove unbroken cells and debris and the supernatant was applied to a HisTrap HP column. After washing the column with the same buffer, the NphB enzyme bound to the Ni²⁺ affinity matrix was eluted with one column volume of Buffer A containing 400 mM imidazole, and then immediately desalted on PD-10 columns (GE Healthcare) equilibrated with mM Tris-HCl, pH 7.5, 5 mM MgCl2, and 10% (v/v) glycerol. The purified NphB protein was quantified and stored at −80° C. prior to use.

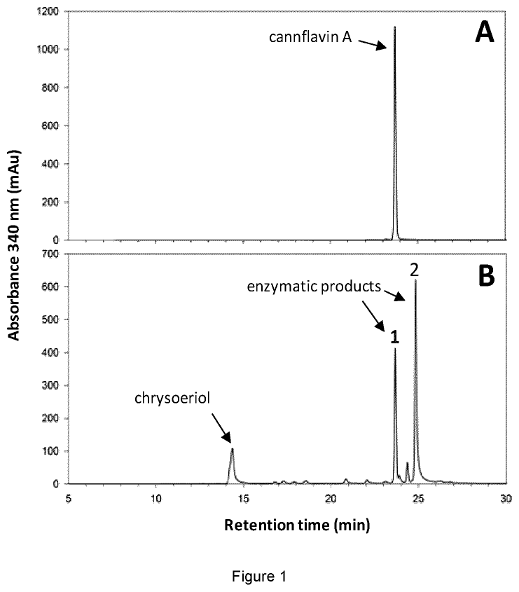

Enzymatic reactions were carried out as follows: purified NphB (200 μg), chrysoeriol (400 μM), GPP (800 μM), MgCl₂ (10 mM), Tris-HCl pH 9.0 (100 mM) were mixed in a total volume of 500 μL and were incubated in a 30° C. water-bath overnight. Reactions were terminated with 20 μL of 20% formic acid and extracted twice with ethyl acetate, dried under N₂ gas, and then resuspended in 200 μL of methanol. Samples were run on an HPLC with a Spherisorb ODS2 reverse-phase column and eluted with a 20 min linear gradient from 45% to 95% methanol in water containing 0.1% formic acid (v/v). The HPLC chromatogram of the product showed a first major peak that eluted at the same time as the cannflavin A standard (FIG. 1) which was collected and processed for mass spectrometry analysis.

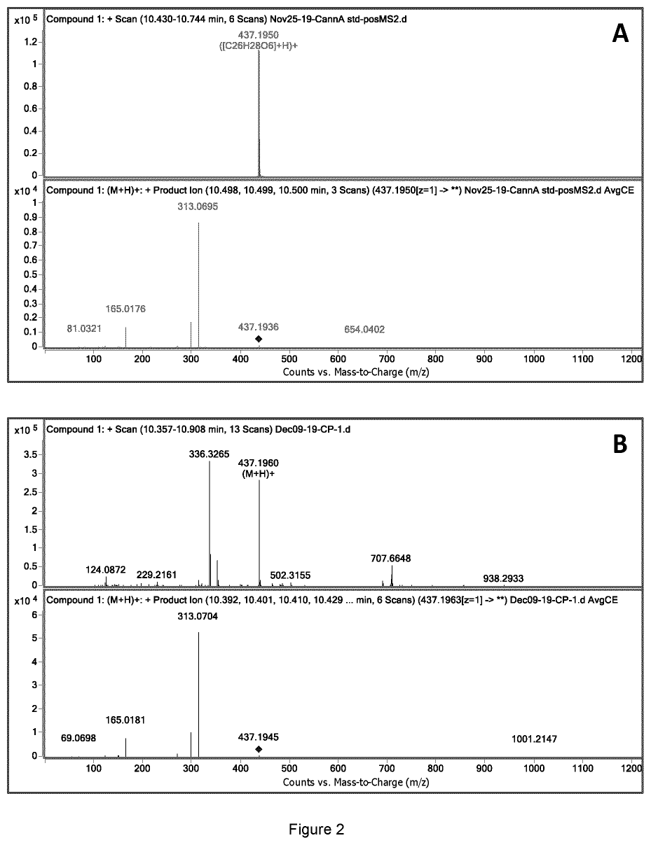

The prenylated products that were produced by NphB in vitro, were purified by HPLC as described above. Samples were evaporated under nitrogen and then re-suspended in methanol prior to liquid chromatography-mass spectrometry (LC-MS) analysis performed on an Agilent 1200 HPLC interfaced with an Agilent UHD 6530 Q-TOF mass spectrometer. A C18 cartridge column (Agilent Rapid Resolution 2.1×30 mm, 3.5 μm) was used at 30° C. with 1:1 water and acetonitrile as solvents, both with 0.1% formic acid. The flow rate was maintained at 0.4 mL/min. The mass spectrometer electrospray capillary voltage was maintained at 4.0 kV and the drying gas temperature at 250° C. with a flow rate of 8 L/min. Nebulizer pressure was 30 psi and the fragmentor was set to 160 V. Nitrogen was used as both nebulizing, drying gas, and collision-induced dissociation gas. The instrument was externally calibrated with the ESI TuneMix (Agilent). The mass-to-charge ratio was scanned across the m/z range of 100-3000 m/z in 4 GHz extended dynamic range positive-ion MS mode. Chromatograms were analyzed within Agilent Qualitative Analysis software B 08.0. Fragmentation patterns of the various parent (molecular) ions were obtained using collision energies of 5, 10 and 20 V, with 20 V being optimal. Those fragmentation patterns obtained from the recovered peak products (geranylation of chrysoeriol) were compared with those of Cannflavin A. Q-TOF mass spectra of a cannflavin A standard shows that the mass spectra of the prenylated product (6-geranyl chrysoeriol) is consistent with the pattern of a cannflavin A standard ([M+H]+ 437) (FIGS. 2A and 2B, upper panels) and mass spectral fragmentation pattern of the enzymatic product from the assay (bottom panel) also resembles that of the cannflavin A standard in (A, bottom panel), indicating that prenylation of chrysoeriol with GPP by NphB produces cannflavin A (FIGS. 2A and 2B, bottom panels).

### Example 2

This example describes a general method for using O-methyltransferases from Cannabis sativa and S-adenosyl-methionine to produce methylated flavonoids, specifically a flavone and a flavonol. Synthetic cDNA sequences of CsOMT6 and csOMT21 were sub-cloned in the pET28a vector system (Novagen) to produce a 6×His-protein fusion and then the construct was introduced into E. coli BL21-CodonPlus (DE3)-RIPL cells. Bacterial cells expressing recombinant proteins were cultured in 1 L of LB media at 37° C. to an OD₆₀₀ of 0.6. Isopropyl-β-D-thiogalactoside (IPTG) was then added to a final concentration of 1 mM and the cells were incubated at 16° C. for an additional 18 h to induce recombinant protein expression. The bacterial cells were collected by centrifugation, re-suspended in 20 mM Tris-HCl, pH 8.0, 500 mM KCl (Buffer A), and then disrupted by sonication. Crude protein extracts were centrifuged at 12,000×g for 10 min at 4° C. to remove unbroken cells and debris and the supernatant was applied to a HisTrap HP column. After washing the column with the same buffer, the enzymes bound to the Ni²⁺ affinity matrix were eluted with one column volume of Buffer A containing 400 mM imidazole, and then immediately desalted on PD-10 columns (GE Healthcare) equilibrated with 50 mM Tris-HCl, pH 7.5, 5 mM MgCl2, and 10% (v/v) glycerol. The purified proteins were quantified and stored at −80° C. prior to use.

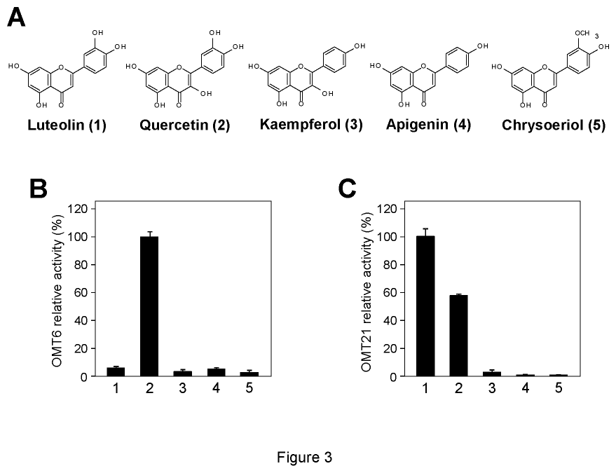

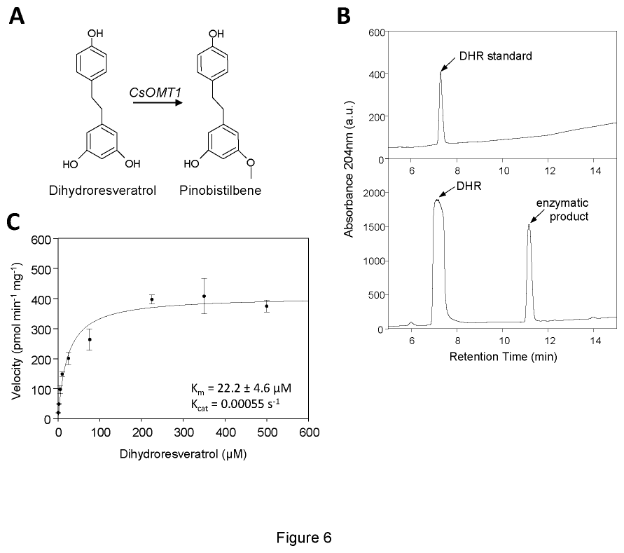

Enzymatic reactions for determining O-methyltransferase activity of CsOMT6 and CsOMT21 were carried out using ˜2 μg of purified recombinant enzyme incubated in a final reaction volume of 100 μL containing 1 mM substrate (luteolin, quercetin, kaempferol, apigenin and chrysoeriol, FIG. 3A) and 6.9 μM S-[Methyl-¹⁴C] adenosyl-L-methionine in 50 mM Tris-HCl, pH 7.5, 5 mM MgCl2, and 10% (v/v) glycerol for 30 min at 37° C. The enzymatic products were extracted with four volumes of ethyl acetate and quantified using a scintillation counter (Model L56500, Beckman). For the identification of reaction products, the assays were scaled up to a final volume of 500 μL using ˜50 μg of recombinant protein, 2 mM substrate and 2 mM S-adenosyl-L-methionine in 50 mM Tris-HCl, pH 7.5, 5 mM MgCl2, and 10% (v/v) glycerol for 60 min at 37° C. The enzymatic products were extracted as above, evaporated to dryness under N₂ gas, and resuspended in 100 μL of methanol. Samples were analyzed by HPLC with a Spherisorb ODS2 reverse-phase column (250 mm×4.6 mm, 5 μm) and eluted over a 20 min gradient from 45% to 95% methanol with 0.1% formic acid (v/v) followed by 100% methanol for min. The eluted products were detected by absorption at the 210-350 nm range and quantified relative to authentic standards. Next, mass spectral analyses of the enzymatic products were performed as described in Example 1. With CsOMT6, preferred methylation of quercetin was detected (FIG. 3B), whereas recombinant CsOMT21 was able to methylate luteolin and quercetin but with less efficiency (FIG. 3C), indicating that the selected OMTs from cannabis present preferred substrate specificity for flavonoid compounds.

### Example 3

This example describes a method for using O-methyltransferases from Cannabis sativa to produce methylated bibenzyls using S-adenosyl-methionine. Similar to what was described in the previous example, cDNA sequences of CsOMT1, CsOMT3, CsOMT5 and CsOMT13 were sub-cloned in the same expression vector and recombinant proteins were produced in E. coli and purified using the same method as above.

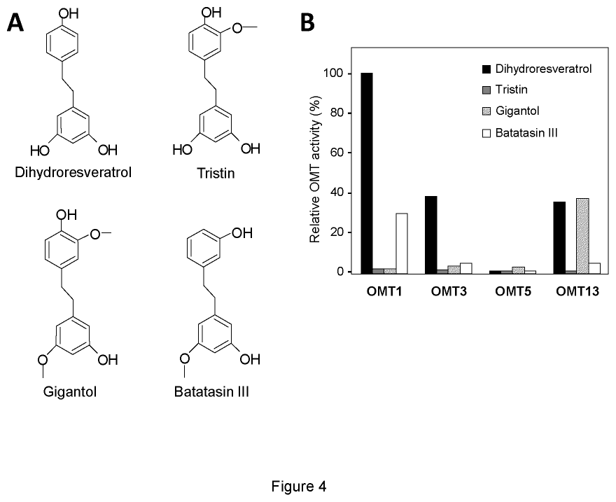

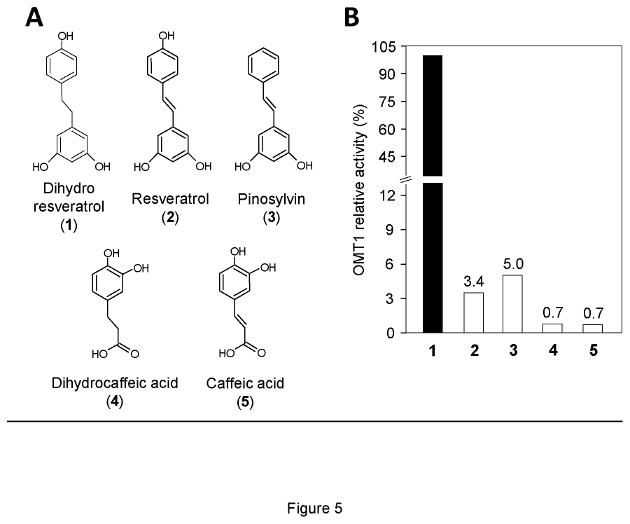

In this example, the activity of these four O-methyltransferases was tested with the bibenzyl compounds dihydroresveratrol, tristin, gigantol and batatasin III as potential substrates (FIG. 4A). The higher O-methyltransferase activity was observed with CsOMT1 when dihydroresveratrol (DHR) was used as substrate. CsOMT1 also displayed activity with batatasin III, although this was about 25% of that with DHR (FIG. 4B). Similar levels of substrate conversion to a methylated form were observed with CsOMT3 with DHR as substrate and CsOMT13 with DHR and gigantol product (FIG. 4B). CsOMT5 did not show any preference with the selected substrates nor produced any specific product. To evaluate whether CsOMT1 can methylate stilbenoids, the oxidized form of DHR, resveratrol, along with pinosylvin and similar phenolic molecules such as caffeic acid and its reduced form, dihydrocaffeic acid (FIG. 5A) were used in similar enzymatic assays. Only residual activity (lower than 5% of that with DHR) was observed with the two stilbenoids tested (FIG. 5B), indicating that CsOMT1 has specific preference to bibenzyl compounds, specifically towards DHR. To further validate and characterize CsOMT1 methyltransferase activity, the identity of the methylated product of the reaction was structurally determined by NMR as pinobistilbene, which indicated that the modification on DHR occurred in position 3 of the ring that has the two hydroxyl groups (ring A, FIG. 6A). FIG. 6B shows a representative chromatogram of the reaction products resolved by HPLC which illustrates the separation of the substrate (DHR) from its methylated form. Enzyme kinetics assays were also performed to characterize CsOMT1 activity, which also confirmed the efficient O-methyltransferase activity of the recombinant enzyme with dihydroresveratrol as substrate (FIG. 6C).

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. A polypeptide encoding a prenyltransferase for prenylating a polyphenol.

2. The polypeptide of claim 1, wherein the prenyltransferase is a microbial prenyltransferase.

3. The polypeptide of claim or 2, comprising or consisting of a polypeptide having at least 80%, 85%, 90%, 91%, 92%, 93%, 94%, 95%, 96%, 97%, 98%, 99%, or 100% sequence identity to the sequence of any one or more of SEQ ID NO: 1-6 and/or a polypeptide listed in Table 1, or a fragment of any thereof.

4. The polypeptide of claim 3, comprising or consisting of the sequence of any one or more of SEQ ID NO: 1-6 and/or a polypeptide listed in Table 1.

5. The polypeptide of claim 4, comprising or consisting of the sequence of any one or more of SEQ ID NO: 1-6.

6. The polypeptide of any one of claims 1 to 5, wherein the polypeptide prenylates the polyphenol using a prenyl donor.

7. The polypeptide of claim 6, wherein the prenyl donor is IPP, FPP, GPP, and/or DMAPP, or a variant or derivative thereof.

8. The polypeptide of any one of claims 1 to 7, wherein the polyphenol is a flavonoid, stilbenoid, and/or bibenzyl, or a derivative thereof.

9. The polypeptide of claim 8, wherein the flavonoid is a flavone, such as apigenin, luteolin, chrysoeriol, chrysin, acacetin, baicalein, baicalin, vitexin, wogonin, orientin, oroxylin A. rutin, or tangeritin; a flavonol such as quercetin, kaempferol, galangin, myricetin, tamarixetin, fisetin, or casticin; a flavanone such as naringenin, hesperetin, pinocembrin, hesperidin, or eriodictyol; a flavanonol such as taxifolin; a flavanol such as catechin, or epicatechin; an isoflavone such as genistein, or daidzein; an anthocyanin such as cyanidin, chrysanthemin, pelargonidin, delphinidin, or malvidin; or any combination thereof.

10. The polypeptide of claim 8, wherein the stilbenoid is resveratrol, piceatannol, pterostilbene, pinosylvin, gnetol, oxyresveratrol, pinostilbene, or any combination thereof.

11. The polypeptide of claim 8, wherein the bibenzyl is a dihydrostilbenoid such as dihydroresveratrol, combretastatin, dihydropiceatannol, dihydrognetol, dihydropinosylvin, gigantol, pinobistilbene, batatasin III, crepidatin, moscatilin, crysotoxine, chrysotobibenzyl, amoenylin, tristin, cumulating, or any combination thereof.

12. The polypeptide of claim 8, wherein the prenyltransferase prenylates the flavonoid to produce 8-prenyl kaempferol, isocannflavin B, cannflavin C, 6-prenylnaringenin, 6-prenylapigenin, neougonin A, neougonin B, and/or kuraridin.

13. The polypeptide of claim 8, wherein the prenyltransferase prenylates the stilbenoid to produce arachidins, isorhapontigenin, rhapontigenin, pawhuskin A, aglaiabbrevin E, amorphastilbol, or longistylins.

14. The polypeptide of claim 8, wherein the prenyltransferase prenylates the bibenzyl to produce canniprene, cannabistilbene, dihydrolongistylins, amorfrutin 1/A, or amorfrutin B.

15. The polypeptide of 9, wherein the prenyltransferase prenylates chrysoeriol using GPP to produce cannflavin A.

16. The polypeptide of claim 9, wherein the prenyltransferase prenylates chrysoeriol using DMAPP to produce cannflavin B.

17. A polypeptide encoding an O-methyltransferase for methylating a polyphenol.

18. The polypeptide of claim 17, wherein the O-methyltransferase is a plant O-methyltransferase.

19. The polypeptide of claim 18, wherein the O-methyltransferase is a Cannabis sativa O-methyltransferase.

20. The polypeptide of any one of claims 17 to 19, comprising or consisting of a polypeptide having at least 80%, 85%, 90%, 91%, 92%, 93%, 94%, 95%, 96%, 97%, 98%, 99%, or 100% sequence identity to the sequence of any one or more of SEQ ID NO: 7-30, or a fragment of any thereof.

21. The polypeptide of claim 20, comprising or consisting of the sequence of any one or more of SEQ ID NO: 7-30.

22. The polypeptide of any one of claims 17 to 21, wherein the polypeptide methylates the polyphenol using a methyl donor.

23. The polypeptide of claim 22, wherein the methyl donor is S-adenosyl methionin, or a variant or derivative thereof.

24. The polypeptide of any one of claims 17 to 23, wherein the polyphenol is a flavonoid, stilbenoid, and/or bibenzyl, or a derivative thereof.

25. The polypeptide of claim 24, wherein the flavonoid is a flavone, such as apigenin, luteolin, chrysoeriol, chrysin, acacetin, baicalein, baicalin, vitexin, wogonin, orientin, oroxylin A. rutin, or tangeritin; a flavonol such as quercetin, kaempferol, galangin, myricetin, tamarixetin, fisetin, or casticin; a flavanone such as naringenin, hesperetin, pinocembrin, hesperidin, or eriodictyol; a flavanonol such as taxifolin; a flavanol such as catechin, or epicatechin; an isoflavone such as genistein, or daidzein; an anthocyanin such as cyanidin, chrysanthemin, pelargonidin, delphinidin, or malvidin; or any combination thereof.

26. The polypeptide of claim 24, wherein the stilbenoid is resveratrol, piceatannol, pterostilbene, pinosylvin, gnetol, oxyresveratrol, or any combination thereof.

27. The polypeptide of claim 24, wherein the bibenzyl is a dihydrostilbenoid such as dihydroresveratrol, combretastatin, dihydropiceatannol, dihydrognetol, dihydropinosylvin, gigantol, batatasin III, crepidatin, moscatilin, crysotoxine, chrysotobibenzyl, amoenylin, tristin, cumulating, or any combination thereof.

28. The polypeptide of claim 24, wherein the O-methyltransferase methylates a flavonoid to produce chrysoeriol, acacetin, tamarixetin, or methylquercetin.

29. In an aspect, the O-methyltransferase methylates a stilbenoid to produce pinostilbene, isorhapontigenin, rhapontigenin, or any combination thereof.

30. The polypeptide of claim 24, wherein the O-methyltransferase methylates a bibenzyl to produce gigantol, tristin, or pinobistilbene.

31. The polypeptide of any one of claims 1 to 30, wherein the polypeptide, variant, or fragment comprises up to about 100, about 150, about 200, about 250, about 300, about 350, about 360, about 370, about 380, about 390, about 400, about 410, about 420, about 430, about 440, about 450, or about 500 amino acids

32. The polypeptide of any one of claims 1 to 31, wherein the polypeptide is synthetic.

33. The polypeptide of any one of claims 1 to 32, wherein the polypeptide is recombinant.

34. A nucleic acid encoding the polypeptide of any one of claims 1 to 33.

35. The nucleic acid of claim 34, wherein the nucleic acid is cDNA.

36. A vector comprising the nucleic acid of claim 34 or 35.

37. A host cell comprising the vector of claim 36.

38. A host cell expressing the polypeptide of any one of claims 1 to 33.

39. The host cell of claim 37 or 38, wherein the host cell is a bacterial cell (e.g., E. coli or Agrobacterium tumefaciens), a yeast cell (e.g., S. cerevisiae), an algal cell, or a plant cell (e.g., Nicotiana spp.).

40. The host cell of any one of claims 37 to 39, in combination with the polyphenol.

41. The host cell of claim 40, wherein the polyphenol is provided in the host cell culture medium.

42. The host cell of claim 40 or 41, wherein the polyphenol is expressed by the host cell.

43. The host cell of any one of claims 37 to 42, in combination with a prenyl donor and/or a methyl donor.

44. The host cell of claim 43, wherein the prenyl donor and/or methyl donor is provided in the host cell culture medium.

45. The host cell of claim 43 or 44, wherein the prenyl donor and/or methyl donor is expressed by the host cell.

46. An expression system comprising the polypeptide of any one of claims 1 to 33; the nucleic acid of claim 34 or 35, the vector of claim 36, or the host cell of any one of claims 37 to 45.

47. The expression system of claim 46, further comprising the polyphenol and a prenyl donor and/or methyl donor.

48. A system for prenylating and/or methylating a polyphenol the system comprising the polypeptide of any one of claims 1 to 33.

49. The system of any one of claims 46 to 48, wherein the polypeptide is in a batch solution.

50. The system of any one of claims 46 to 49, wherein the polypeptide is immobilized in a support matrix.

51. The system of any one of claims 46 to 50, wherein the polypeptide is in a cell.

52. The system of any one of claims 46 to 50, wherein the system is cell-free.

53. A method for prenylating and/or methylating a polyphenol, wherein the method comprises contacting the polyphenol with the polypeptide of any one of claims 1 to 33.

54. The method of claim 53, carried out in the system of any one of claims 46 to 52.

55. The method of claim 53 or 54, wherein the method is a recombinant method comprising expressing the polypeptide of any one of claims 1 to 33 in a cell in the presence of the polyphenol and a prenyl donor and/or methyl donor.

56. The method of any one of claims 53 to 55, in combination with a synthetic chemical catalysis method.

57. The method of any one of claims 53 to 56, comprising a single synthesis step.

58. The method of any one of claims 53 to 57, wherein the method is carried out in combination with an enzymatic reaction.

59. The method of any one of claims 53 to 58, comprising a combined enzymatic O-methylation and prenylation step.

60. A method of producing cannflavin A, cannflavin B, isocannflavin B, the method comprising carrying out a combined enzymatic O-methylation and prenylation of a flavonoid.

61. A method of producing a longistylin, the method comprising carrying out a combined enzymatic O-methylation and prenylation of a stilbenoid.

62. A method of producing canniprene, cannabistilbene, dihydrolongistylin, amorfrutin 1/A, or amorfrutin B, the method comprising carrying out a combined enzymatic O-methylation and prenylation of a bibenzyl.

63. A synthetic chemical catalysis method of producing cannflavin A and/or cannflavin B, the method comprising using GPP and DMAPP in a single synthesis step from chrysoeriol or in combination with an enzymatic reaction such as the O-methylation of luteolin.

64. A prenylated and/or methylated polyphenol produced by the method of any one of claims 53 to 63.

65. The polyphenol of claim 64, wherein the polyphenol is substantially pure, for example, at least about 95%, about 96%, about 97%, about 98%, about 99%, about 99.5%, or about 99.9% pure.

66. The polyphenol of claim 64 or 65, wherein the polyphenol is cannflavin A and/or cannflavin B.

67. A cosmetic composition comprising the polyphenol of any one of claims 64 to 66 and at least one cosmetically acceptable carrier.

68. A pharmaceutical composition comprising the polyphenol of any one of claims 64 to 66 and at least one pharmaceutically acceptable carrier.

69. A natural health product comprising the polyphenol of any one of claims 64 to 66, such as a supplement, beverage, or food.

70. Use of the polyphenol of any one of claims 64 to 66 in a cosmetic, pharmaceutical, or natural health product.


SEQUENCE LISTING (30 sequences)

SEQ ID NO: 1 - protein, 307 aa, Streptomyces sp. CL190 [sequence omitted]

SEQ ID NO: 2 - protein, 295 aa, Streptomyces coelicolor
MPTGRTTDLALFLSDLEAYAKLAEVTFDKRAVEQVVDVFAEQFATGTITVRTTTHEAANRSVNFRYMYPDSPHDPVEIARAHGLLPDADPAVMSLLAEVTEKIPLWWGLDASVGHGVQKVWAFFEQPLEFGEIASLENTPHSLRDHRERFGEARIDRFAIMGFDFRDNTTNLYSEMVSPGYFEQEEVARMIRDVGSLPPDNEEIERCRGAINVYYTFDWNSPQARRLCFAVPSRDGEFPSHLHPLAARFAAEAPVQAERRELIFNPTFGARGSYLKMEADYTGDAASRVFGYWNR

SEQ ID NO: 3 - protein, 324 aa, Streptomyces roseochromogenus subsp. oscitans
MPALPIDQEFDCERFRADIRATAAAIGAPIAHRLTDTVLEAFRDNFAQGATLWKTTSQPGDQLSYRFFSRLKMDTVSRAIDAGLLDAAHPTLAVVDAWSSLYGGAPVQSGDFDAGRGMAKTWLYFGGLRPAEDILTVPALPASVQARLKDFLALGLAHVRFAAVDWRHHSANVYFRGKGPLDTVQFARIHALSGSTPPAAHVVEEVLAYMPEDYCVAITLDLHSGDIERVCFYALKVPKNALPRIPTRIARFLEVAPSHDVEECNVIGWSFGRSGDYVKAERSYTGNMAEILAGWNCFFHGEEGRDHDLRALHQHTESTMGGAR

SEQ ID NO: 4 - protein, 323 aa, Streptomyces niveus
MPALPMNQEFDRERFRVDLRATAAAIGAPVTPRVTDTVLETFRDNFAQGATLWKTTSQPGDQLSYRFFSRLKMDTVGRAVDAGLLDGTHPTVPIVEDWSDLYGGTPVQSADFDAGRGMAKTWLYFGGLRPAEDILSVPALPAPVQARLKDFLGLGLAHVRFAAVDWRHRSANVYFRGQGPLDTAQFARVHALSGGTPPAADVVAEVLAYVPEDYCVAITLDLHTGAIDRVCFYALKVPKDARPRVPARIATFLEVAPSHDPEECNVIGWSFGRSGDYVKAERSYTGNMTEILSGWNCFFHGEEGRDHDLRALQDTGSITGGAR

SEQ ID NO: 5 - protein, 307 aa, Streptomyces sp. KO-3988
MPGTDDVAVDVASVYSAIEKSAGLLDVTAAREVVWPVLTAFEDVLEQAVIAFRVATNARHEGDFDVRFTVPEEVDPYAVALSRSLIAKTDHPVGSLLSDIQQLCSVDTYGVDLGVKSGFKKVWVYFPAGEHETLARLTGLTSMPGSLAGNVDFFTRYGLADKVDVIGIDYRSRTMNVYFAAPSECFERETVLAMHRDIGLPSPSEQMFKFCENSFGLYTTLNWDTMEIERISYGVKTENPMTFFARLGTKVEHFVKNVPYGVDTQKMVYAAVTSSGEEYYKLQSYYRWRSVSRLNAAYIAARDKEST

SEQ ID NO: 6 - protein, 299 aa, Streptomyces anulatus
MSESAELTELYSAIEETTRVVGAPCRRDTVRPILTAYEDVIAQSVISFRVQTGTSDAGDLDCRFTLLPKDMDPYATALSNGLTAKTDHPVGSLLEEVHRQFPVDCYGIDFGAVGGFKKAWSFFRPDSLQSASDLAALPSMPSGVSENLGLFDRYGMTDTVSVVGFDYAKRSVNLYFTGASPESFEPRGIQAILRECGLPEPSDELLRFGEEAFAIYVTLSWDSQKIERVTYSVNTPDPMALPVRIDTRIEQLVKDAPLGSAGHRYVYGVTATPKGEYHKIQKYFQWQSRVEKMLTADAG

SEQ ID NO: 7 - protein, 368 aa, Cannabis sativa
MEADGEDAVLRGQVEIWKYMLSFADSMALKCAVELQLADIIHSHTSPITLSQIASAIPGATSPDLSCLARIMRLLVRRRIFTQHQKPKSDGEEEEALYGPTHSSRWLLTKTNDHDQLTLAPMILMENDPRLMAPWHCFSRCVKEGGVAFKKAHNGQSIWEFGAENPEINKLFNDAMECTAKVVMKAILSHYSDGGFSDIKSMVDVGGGTGGSISEIVRSYPHIKGINFDLPHVIATAPPYSGVSHVGGDMFRSVPTADAIFMKWILHDWSDEDCVKILKNCRKAIAEESGKVIIVESVLEEESNNNNNNNEVFGDTALMLDLVMVAHTTGGKERTQKQWKTILEQGGFPRYNFIKIKALPSIIEAYPN

SEQ ID NO: 8 - protein, 363 aa, Cannabis sativa
SELLFQAQTHLYNHTLSYISSMCLKCAIELGIPDIINNHGQPHIPLPQLVSSLRLPPTKTDILRRLMRPLVHFGYFTTTKVVINSQNKEEEEEEVDAYGLTSSSKLLFVNNNGNNKIPSMSTIVCLQLDQAFMTPWHSLGNWLRKDEATTLFESAHEMSFWEYTSKNTKFGHLFNEAMADDSKLMLKLVIEDVKPVFEGLTSLVDVGGGTGEVCKILTQVFPHLKCSVLELSHVVANLPNAQNLKFIEGDMFQAIPPANAVLLKWILHNWSDEECVTILKKCREAIASNEGGKVVIIDVVINSKKDEHEVNEAKISFDLMMMVLFNGRERSQKEWENLFFKAGFTRYKITPIFGLESLIEVFP

SEQ ID NO: 9 - protein, 370 aa, Cannabis sativa
MASAVKGAILAIDNEASFQDQAEIWKYMLNFGDSMALKCAVELRLPDILHSADGPMTLSEIAAAIPNAPSPQASHLFRVLRLLVRRKIFTSEEDKVSGETLYGPTKLSSWLLHEPAPTSDSDASIMTLAPMLIMENHPWHVDPWHKLSEFIREGGLAPFEKEHGYMFDFAAKNPLYNKLLNNAMACTARITIRTLLSHCGDDLFNGVGSVVDVGGGTGRFISEIVKSYPHIKGINFDRSHVISTAPAYPGVTHIGGDMFQEVPSADAVVTKWVLHDWGDELCVKLLKNCRKAIPEKGGKVIIVDIVVEADGEGLFDDTGVVFDVLMLAHNTGGKERTEKEWKSLLDQSGFPRYKITKIPALQSVIEAYPN

SEQ ID NO: 10 - protein, 375 aa, Cannabis sativa
MMSSINDNTITTTQQLSLGYVNLYKHMLSYASSMALKSAVELGIPDIIFNKGKAQTISLHELVSALQIPPSRTNFLRRVMRVLVHSGIFTNEKGYNDDKEEEEVYGLTPSSKLLLTNGNNSEVPSVGPFVLSVLEPVTVTAFHLIGNWLKNEDSPATPFHLANDDGLGLYEYWGKNTDGFGDRLNEGMESDSGTLKFVLKNFKSTFEGITTLVDVGGNTGTMCKMLIEAFPHLKCSVFDLPYVVEANSHNNTENLKFIEGDMFQTIPEADAILFKLVLCGCSDDESIKILKNCREAISRNGKGKVLIIENNVINSEKDELLELEAKLYFDMLLLASVTGRERSKKDWENIFFQAGFTHYEITPMFGLEALIEVFP

SEQ ID NO: 11 - protein, 369 aa, Cannabis sativa
MDALSRDQAEIFEHMFSFVDSMALKCAVELRIADIIHSQDCPISLTQIASKIITNSHNSPMISSSPDNNTMLYLNRIMTSLVRKKIFTAQYDHDQNNNQTVLYYGVTSKSRWLLRDAKPSLAPLILMENHPIQMAPWHYFSHIIKDQEGSATAYEKAHGCGIFELASVNGELNKIFNDGMACLGEMVMGAILPAYDVFGCMGSLVDVGGGIGGDLAEIVKSHPHIKGINFDLPHVTATAPESNGVTHVAGNMFESVPSADAIFIKWVLHDWCDEECVKILRNCRKAIPEKNGKVIIVEIVLKDSSQNKENDDVFDETRMIFDMVMMAHTCRGKERTEFEWKKLLEEAGFPRYKITKIPAIPSIIEAYPF

SEQ ID NO: 12 - protein, 362 aa, Cannabis sativa
MAPTQISEELEASLFAMQLAGTSSILPMVLKTALELDLLEIIAMAGPNAFLSPSDIAAQLPTNNPNASMMLDRMLRLLASYNVLTYLLRDKVTSDGKVLVERLYGLAPLSKFLTKNEDGASIAPLCLMVQDKVFMESWYHLKDAILDGGIAFDKAHGMPAFKYNQIDKRFNKIFNKGMFDHSSITMKKILETYKGFEGLNSMVDVGGGSGAVLSMIVTKYPSIKGINFDLHHVIEDAPPFPGVAHVGGDMFVSVPKGDAIFIKWICHDWSDEDCLKLLKNCYDAVPHHGKVIVAEFILSVAPDSSLAAKCTAHSDMIMLVDHGGKERTQKEFEELAKAAGFKGFKVVCNAFNTYIMEFLKTN

SEQ ID NO: 13 - protein, 377 aa, Cannabis sativa
MAVETHKDELIWIPKEDEEERARVDIYKYIFGFVEMAVVKCAIELGIADAIESHGRPMSLLELSSALGCAAPALHRIMRFLTNRKLFKEIRINENVQDSEQPSLYAQTALSRLILRSGEKSMATFVLMESSPPMLAPWHGLSARVKTEVDDSSAPSPFEVANGKDVWSYAAANPGHSQLINEAMACNARVTVAAILDGCLDVFDGIGTIVDVGGGNGTALRMLVRACPWIRGINFDLPHVVSVALKSEGVEHVGGDMFKFVPKADAAFLMSVLHDWEDDECIQILKKCREAIPGDKGKVIMVECVIEENNNNVEEKHEELELKDVGLFLDMVMIAHTNKGKERTLEEWAYVLAQAGFNRYNIRAINAIYSIIEAFPN

SEQ ID NO: 14 - protein, 384 aa, Cannabis sativa
MAKMQEGGDHHDELTCRAKEEDEQEEERARIDIYKYVFGFVEMAVVKCAIELGIADTIECHGRPMSLKELSSALGCTPHNIHRIMRFLVNRRIFKEIKNDIVNDEGAGTLYVQTSLSRLLIKSGERSMASFVLMESSKPMLAPWHCLSSRLKAEVIDNSLTPFEEANGQDLWSYTAANPEHSQLLNEAMACNARVTVAAILDSCLEVFDGVGSIVDVGGGNGTAMQLLVKGCPWIKEGILFDLPHVVSVALKSDRVVHVGGDMFDSVPKADAAFLMWVLHDWEDKDCIQILKNCREAISEKGKVIIVESVIENNKEQNNGMKKDELEFKDVGLFLDMVMMAHTNKGKERTLDQWVYVLHQAGFTRYNVRSIKGAISSLIEAFPI

SEQ ID NO: 15 - protein, 373 aa, Cannabis sativa
MEKLKSFSYLNNNIDLAMNEENNTKLLGAQAHIWNQIFNFINSMCLKCAVELGIPDIINNYGKPMTISQLTLALSINKNKFHCLYRLMRLLTHSGFFALEKVEIEGEKEEEGYVITEASKLLLKDNPMSVTPFLLLVLNPILTKSFDVLDTWFQNDSPTPFDTANGRTFWDYGSHEPKLVQLFNDGMASDARLVTSVVIEKCKGVFEGVERLVDVGGGTGTVAKSIATAFPQIECSVLDLPHVVADLEDENNLKFIGGDMFVEIPTADVVLLKWILHDWNDEESVKILKNCREAVYKSKKKSGKLIIIDMNIKNDNNENSFETQLFFDMLMMALVSGRERNEKEWSKLFKDAGFSRYKITPILGLRFVIEVYP

SEQ ID NO: 16 - protein, 377 aa, Cannabis sativa
MDGIQEGDHHDELTLRLNEKEEEERARIDIYKYVFGFVEMAVVKCAIELGIADTIESHGRPISLLDLSSALSCNPHNLHRIMRFLVNRRIFKEIKNDTVNDKGCLYVQTSLSRLLIKSGERSMASFVLMESSNPMLAPWHGLSARVKAEATDALTPFEAANGVDVWSYAAANPDHSQLINEAMACNARVTVAAILNGCLDVFDGVGSIVDVGGGNGTTLQLLVKGCPWINQGINFDLPHVVSVALKSDGVVHVGGDMFDSVPKADAAFLMWVLHDWGDQECIQILKKCKEAIPEKGKVIIVESVIENNKLEENVMKKELELKDVGLFLDMTMMAHTNKGKERSLDEWVYVLHQAGFTRYNVRSIDGAVSSVIEAFPA

SEQ ID NO: 17 - protein, 373 aa, Cannabis sativa
MGSISENTTTLELSQGYVNVYKHMLSYASSMALKCAIQLHIPDIIFTKGKDQTITLPELASALQIPPSRISCLRRVMRVLVHSGIFSNKNQHDDDKTEEEEVYGLTSSSKILLTGGNNNGVPSVGGYVLAVLEPVTVTAFHLIGSWLKKESPRTPFHLANDEGLSLYEYWGKNIDGFGDRLDEGMESDSGVLKFVLKDLKSSFEDITTLVDVGGNTGSMCMMLIEEFPHLKCTVFDLPYAIEANSHNSTSNLKFVEGDMFQSTFPEADAFLLKSVLSGCSDEECVKILKNCGEAISRNGGGKVMVIDNNVINTKNDEAAELEAKLYFDMLFMTALTGRERTKKDYENIFYQAGFTRIKITRMFGLKSLIEGFL

SEQ ID NO: 18 - protein, 367 aa, Cannabis sativa
MTTPTQMSEELEANYLFAMKLASATVLPMVLKTALELGLLEIIVMAGPGAFLSPSNIVAQLPTKNPNAPVMLDRMLRLLASYNVLTYSIRDGERLYGMTPLSKFLTKNEGGLSIAPLCHMDQDKVIIDCWYHMKDAVLDGGIPFNKAHGMPIFEYTQRDQRLNKIVNRAMSTLSTIIMKNILETYNGFKGLNSIVDVGGGTGATLSMIIAKYPSIKGINFDLHHVIQHAPPLPGVEHVSGDMFVSVPKGDAIFMKRICHDWSDEECLKLLKNCYDALDDDGKVIVEELIVPAAPDSSPSTKNSFHYDILMMVNLNGKERTQKEYEQLAMEAGFKAFKIHCIAFNSYIMEFLAKGPKVFWSVRVPPLL

SEQ ID NO: 19 - protein, 356 aa, Cannabis sativa
MESSQLRGQELICQLIFSYYNTMALKCAVELRIADIIHSHGKPITISQIASDIQSNSNSKSPINIDNLFRIMRILVRKGVFIEHHDDDHGDSTISLYGLCDSSRCLLWDFDSSLVPFILLNTHPLMMASSHNFGKSVIGDKGNPFENDQDVWSFASNNPIFNKLFNDAMISGSHMVLRHVLSTYKDSFNCIKGTMVDVGGGVGQVISEIVKSHRHIKGINFDLPHVIATAPTYDGVTHVGGDMFESIPSADVVFLKWIIHGWNDDACVKILKNCRKAIGEKKNGKIIIVDMVLDPNSNEIFQETRLAMDLVMLANSNNGKERTELEWKKLLNEAGFSRYKITKNQNILDIIEAFPF

SEQ ID NO: 20 - protein, 373 aa, Cannabis sativa
MGSINENTITTTQELSQGYVNLYKHMLSYASSMALKSAVELGIPDIIFTKGKAQTISLHELVSALQIPPSRTNFLRRVMRVLVHSGIFTNEKGYNDDKEEEEVYGLTPSSKLLLTGGKNNGVPSVGPYVLAVLEPVTVTAFGSIGNWLKKESPTTPFHLANDEGLSLYEYWGKNTDGFGDRLNEGMESDSGVLKFVLKDFKSVFEGITTLVDVGGNTGLMCKMLLEAFPHLQCSVYDLAYAVDANSHNNTQNLKFIEGDMFQTVPQADAILFKCVLSGCSDEECTKILKNCRDAISRNGGGKVLIIDNNVINSKTEDHLAMETLLYFDMLMMTALTGRERTKKDWEKIFFEAGFSSCKVTPMFGVKTLIEVFP

SEQ ID NO: 21 - protein, 363 aa, Cannabis sativa
MGSELEGTTEVVVDLKRKQEEESFCYAAQLLNTNVLTKSLQTTIELGIFDIIAKAGEGGKLSAREIVAQLPTNNPDAPMMVDRILRMLASYSVLVCSVVADDQRAYSLNNVSKCFVTNEDGVSLGPLMLLLEDKVFSDSWSQLKGAILEGGIPFNRFHGMNAFEYPALDSRFNKVFNRAMQSMTTMLAKQTIESYKGFENLKQLVDVGGGLGVTLKEITSTYPHIKGINFDLPHVVQHAPSYPGVEHVGGDMFESVPSGDAIFMKWILHDWSDEQCVKVLKNCYKAIPENGKVIVMEGLLPMLPEASYGDNIMSKTDVLMMTQNPGGKERSKQEFQALASGAGFNGIRFECCVSGFWIMEFFK

SEQ ID NO: 22 - protein, 363 aa, Cannabis sativa
MAPPSEELANTPQIVNDERKQEEENFAYAAQLVNSSVLSMSLQSAIELGVFDIIAKAGDAAKLSAQDIVAQMPTTNPDAPRMLDRILRMLASHSVLACSLENEDLRVYCLNDVSKLFVTNEDGVSLGPLMLLQDKVFLDSWSQLKGAILEGGIPFNRVHGMHAFEYPSLDQKFNQVFNKAMYNQTTLVLKKILEVYKGFENLEKVVDVGGGLGGTLNQITSKYPHIKGINFDLPHVVEHAPSYPGVEHVGGDMFESVPTGAIFMKWILHDWSDEHCLKLLKNCYKAIPDNGNVIVMEAILPTIPETNSADRCTSQTDVLMMTQNPGGKERSKQEFQALASGAGFNGIRFECCVSGFWIMEFFK

SEQ ID NO: 23 - protein, 361 aa, Cannabis sativa
MMGSDQLEVIVDLKRPKQEESFCYALQLLSTNILIKSLQATVELGIFDIIAKAGEGSKLSAAEIVAQLPTNNPDAVMMVDRILRMLAGHSVLTCSVVADNPRVYSHNTVSKCFVTDEDGVSLGSLISLLDDKVYSDSWSQLKGAILEGGIPFNRLHGMNSFEYTALDSRFNKVFNRAMQSMTTMIAKQTIESYKGFENLKQLVDVGGGLGVTLKEITSTYPHIKGINFDLPHVVQHAPSYPGVEHVGGDMFESVPSGDAIFMKWILHDWSDEQCVKVLKNCYKAIPENGKVIVMEGLLPMLPEASYGDNIMSKTDVLMMTQNPGGKERSKQEFQALASGAGFNGIRFECCVSGFWIMEFFK

SEQ ID NO: 24 - protein, 365 aa, Cannabis sativa
MAPPSEELANTPQIVNDERKQEEENFAYAAQLVNSSVLSMSLQSAIELGVFDIIAKAGDAAKLSAQDIVAQMPTTNPDAPRMLDRILRMLASHSVLACSLENEDLRVYCLNDVSKLFVTNEDGVSLGPLMSLLQDKVFLDSWSQLKGAILEGGIPFNRVHGMHAFEYPSLDQKFNQVFNKAMYNQTTLVLKKILEVYKGFENLEKVVDVGGGLGGTLNQITSKYPHIKGINFDLPHVVEHAPSYPGVEHVGGDMFESVPTGDAIFMKWILHDWSDEHCLKLLKNCYKAIPDNGNVIVMEAILPTIPETNSADRCTSQTDVLMMTQNPGGKERSKKEFLALATGAGFSGIRFECFVCNFMIMEFYK

SEQ ID NO: 25 - protein, 372 aa, Cannabis sativa
MEKSRNSSSHVDLVVNEDNNTKLLRAQAHIWNHICKFINSMSLKCAIELGIPDIVNNHGKPMTISQLTLALPINKNKSHCLYRLMRLLTHSGFFALEKTEIKGEEEEEGYVITEASKLLLKDNPMSVTPLLLVLLDPTLTKPYDVLSTWFRNDDSTPFVTTNGMAIWDYYSHEPKLAQSFNEAMASDARLVTSVLIEKCKGVFEGVDSLVDVGGGTGTVAKSIATTFPQIQCSVLDLPHVVAGLQGEKNLNFIAGDMFVEVPTAQVVLLKWILHDWSDENSVKILKKCKEAITKSGKKIGKVVVIDMIIENEKGEIDDESYETQLFMDMTMTLVSGRERNEKELSKLFKDAGFSHYKITPILGLRSLIEIYP

SEQ ID NO: 26 - protein, 377 aa, Cannabis sativa
MEKLKSFRHLNNNIDLVLNEENSIELLRAQGHIWNQIFNFINSMSLKCAIQLGIPDIINNYGKPMTISQLKLALPINQKKSSCVYRLMRILTHSNFFALQKVEGREGEEEEEGYVITDASKLLLKDNPMSVTPFLLAMLDPVITKPWDFLSNWFQNDDPTPFDTANGMTFWDYGSHQPNLARFFNDAMASDARLVTSVVIEKCRWVFEGVESLVDVGGGTGTVATTIATSFPQIQCSVLDLPHVVADLQGANNLVNFIGGDMFVEVPPAEVVLLKWILHDWNDEESVKILKKCKEAITKNNKKGGKVIIIDMKVENEKDEDDESYETQLFFDMLMMALVTGKERNEKEWAKLFKDAGFSDYKITPILGLRSLIEVYP

SEQ ID NO: 27 - protein, 370 aa, Cannabis sativa
MGSTGIETQMTPTQISDEEANLFAMQLASASVLPMVLKAALELDLLEIIAKAGPGAFLSPSDIAQQLPTQNPDAPVMLDRMLRLLASYNVVTYSLRERETAEEEGKVERLYGLAPVSKYLTKNEDGVSIAPLCLMNQDKVLMESWYHLKDAVLDGGIPFNKAYGMTAFEYHGTDQRFNKIFNRGMSDHSTITMKKILETYKGFEGLNSIVDVGGGTGAVVNMIVSKYPTIKGINFDLPHVIEDAPPLTGVEHVGGDMFVSVPKGDAIFMKWICHDWSDEHCLKFLKNCHAALPEHGKVIVAECILPVAPDSSLATKSTVHIDVIMLAHNPGGKERTEKEFEALAKGAGFKGFKVHCNAFNTHIMEFLKTI

SEQ ID NO: 28 - protein, 367 aa, Cannabis sativa
MNLIMGEGELVSCRELVEAQELIYNCSLSHIKPMSLKCAIELGIPDIIHNHGQPITLSKLISSLPIHPSKAHCIHRLMRILVHFGFFTTQLLLPQQQEETYSLTLASKLFLKDCPIKATPFFLVQLNPLLLKPWHFLSTWLQKGEDDDDHPSTPFEMANGINFWDGVGNDPMVKYMFTEAMATDSYLMSKVIVEEGKEVFEGLSSLVDVGGGTGIMANAIVEAFTNIKCTVLDLPYIVADLKGTHNLNFVEGDMFKKIPSANAVLLKWTLHDWNDEEVVVILKKCREAIWSKDKGGKVIVIDMVIDDDEEPKSSVETQLCFDMLMMVNLTGKERNEKEWENLFLAAGFSHYKINPIVGFRSLIEVFP

SEQ ID NO: 29 - protein, 376 aa, Cannabis sativa
MEKLKSFRHLNNNIDLVLNEENSIELLGAQGHIWNQIFNFINSMSLKCAIQLGIPDIVNNYRKPMTISQLVLALPINQKKSPCVYRLMRILIHSGFFALQKVEGGGEGEEEEGYVITDASKLLLKDNPMSVTPFLLSMLDPVMTKPWDFLSNWFQNDDPTPFDTANGMTFWDYGSHQPNLARFFNDAMASDARLVTSVVIEKCKWVFEGVESLVDVGGGTGTVATSIATNFPQIQCTVLDLPHVVADLQGGNNLNFVGGDMFVEVPTAEVVLLKWILHDWNDEESVKILKKCKEAIMKSKKKGGKVIIIDMKVENEKDEDDESYETQLFFDMLMMTLLTGKERNEKEWAKLFKDAGFSDYKITPILGLRSVIEVYP

SEQ ID NO: 30 - protein, 371 aa, Cannabis sativa
MQKGQKGCQINQIPMSIERNNVEEDESFFYAVELRSSVVLPMSLYATIELGVFEILAKAGDGAKLSSSDIASHLPTENPDAPMMLDRILTLLASHSVLDCVVVGEGSSMRKLYSLSPVSKHFLPKEDGVSSHALMKLGLDKVSLESWFELKNAVLEGGTSFKRAHGMNVFEYGKSDSRFGEVFNSAMYNQAKIVTKKIIESYKGFENNIKTLVDVGGGFGVTVSLIVSKYPQIKAINFDLPHVIKNAPTYPGVEHVGGDMFEKIPNGDAIFMKWILHDWNDEDCVKILKKCYEAIPSNGKVIVVDMVVPIMAETTHKAKSIFQLDLVMLSQNPGGKERNQHEFQAIANAAGFSTINFACSIENVKVIEFIK